(19) United States
(12) Patent Application Publication
(10) Pub. No.: US 2007/0197541 A1
(43) Pub. Date: Aug. 23, 2007

(54) Biaryl heterocyclic amines,amides, and sulfur-containing compounds and methods of making and using the same

(76) Inventors: Adegboyega K. Oyelere, Marietta, GA (US); Joel A. Goldberg, Philadelphia, PA (US); Alia Orbin, Macungie, PA (US); Joseph M. Salvino, Chester Spring, PA (US); Jiacheng Zhou, Hockessin, DE (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/566,149

(22) PCT Filed: Jul. 28, 2004
(86) PCT No.: PCT/US04/24334

§ 371 Date: Apr. 18, 2007

Related U.S. Application Data
(60) Provisional application No. 60/490,855, filed on Jul. 29, 2003.

(51) Int. Cl. A61K 31/506; A61K 31/501; A61K 31/497; A61K 31/4439; C07D 413/02

(52) U.S. Cl. 514/252.02; 514/252.05; 514/252.11; 514/255.05; 514/256; 514/341; 544/238; 544/333; 544/295; 544/357; 546/271.4; 546/272.1

(57) ABSTRACT

The present invention relates generally to the field of anti-infective, anti-proliferative, anti-inflammatory, and prokinetic agents. More particularly, the invention relates to a family of biaryl heterocyclic amines, amides, and sulfur-countering compounds that are useful as such agents agents.

### RELATED APPLICATIONS

This application claims the benefit of and priority to U.S. Patent Application No. 60/490,855, filed Jul. 29, 2003, the entire disclosure of which is incorporated by reference herein.

### FIELD OF THE INVENTION

The present invention relates generally to the field of anti-infective, anti-proliferative, anti-inflammatory, and prokinetic agents. More particularly, the invention relates to a family of biaryl heterocyclic amine and amide compounds that are useful as therapeutic agents.

### BACKGROUND

Since the discovery of penicillin in the 1920s and streptomycin in the 1940s, many new compounds have been discovered or specifically designed for use as antibiotic agents. It was once believed that infectious diseases could be completely controlled or eradicated with the use of such therapeutic agents. However, such beliefs have been shaken by the fact that strains of cells or microorganisms resistant to currently effective therapeutic agents continue to evolve. In fact, virtually every antibiotic agent developed for clinical use has ultimately encountered problems with the emergence of resistant bacteria For example, resistant strains of Gram-positive bacteria such as methicillin-resistant staphylocci, penicillin-resistant streptococci, and vancomycin-resistant enterococci have developed, which can cause serious and even fatal results for patients infected with such resistant bacteria. Bacteria that are resistant to macrolide antibiotics, i.e., antibiotics based on a 14- to 16-membered lactone ring, have developed. Also, resistant strains of Gram-negative bacteria such as H. influenzae and M. catarrhalis have been identified. See, e.g., F. D. Lowry, “Antimicrobial Resistance: The Example of Staphylococcus aureus,” J. Clin. Invest., 2003, 111(9), 1265-1273; and Gold, H. S. and Moellering, R. C., Jr., “Antimicrobial-Drug Resistance,” N. Engl. J. Med., 1996, 335, 1445-53.

The problem of resistance is not limited to the area of anti-infective agents, because resistance has also been encountered with anti-proliferative agents used in cancer chemotherapy. Therefore, there exists a need for new anti-infective and anti-proliferative agents that are both effective against resistant bacteria and resistant strains of cancer cells.

In the antibiotic area, despite the problem of increasing antibiotic resistance, no new major classes of antibiotics have been developed for clinical use since the approval in the United States in 2000 of the oxazolidinone ring-containing antibiotic, N-[[(55)-3-[3-fluoro-4-(4-morpholinyl)phenyl]-2-oxo-5-oxazolidinyl]methyl acetamide, which is known as linezolid and is sold under the tradename Zyvox®D (see compound A). See, R. C. Moellering, Jr., “Linezolid: The First Oxazolidinone Antimicrobial,” Annals of Internal Medicine, 2003, 138(2), 135-142.

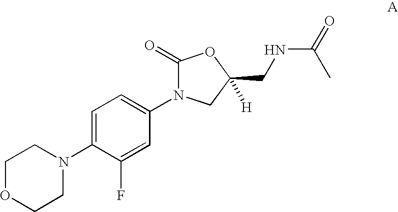

Linezolid was approved for use as an anti-bacterial agent active against Gram-positive organisms. Unfortunately, linezolid-resistant strains of organisms are already being reported. See, Tsiodras et al., Lancet, 2001, 358, 207; Gonzales et al., Lancet, 2001, 357, 1179; Zurenko et al., Proceedings Of The 39^{th} Annual Interscience Conference On Antibacterial Agents And Chemotherapy (ICAAC); San Francisco, Calif., USA, (Sep. 26-29, 1999). Because linezolid is both a clinically effective and commercially significant anti-microbial agent, investigators have been working to develop other effective linezolid derivatives.

Notwithstanding the foregoing, there is an ongoing need for new anti-infective and anti-proliferative agents. Furthermore, because many anti-infective and anti-proliferative agents have utility as anti-inflammatory agents and prokinetic agents, there is also an ongoing need for new compounds useful as anti-inflammatory and prokinetic agents.

### SUMMARY OF THE INVENTION

The invention provides a family of compounds useful as anti-infective agents and/or anti-proliferative agents, for example, chemotherapeutic agents, anti-microbial agents, anti-bacterial agents, anti-fungal agents, anti-parasitic agents, anti-viral agents, anti-inflammatory agents, and/or prokinetic (gastrointestinal modulatory) agents, having the formula: 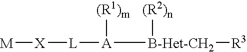
or a pharmaceutically acceptable salt, ester or prodrug thereof, wherein A and B are selected from the group consisting of phenyl, pyridyl, pyrazinyl, pyrimidyl, and pyridazinyl; Het-CH₂—R³ is selected from the group consisting of: 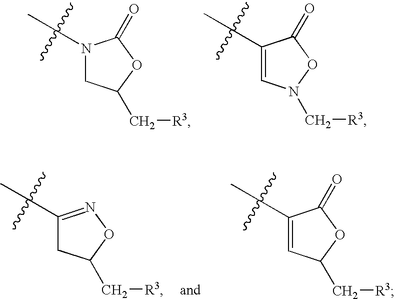
L is an optionally substituted C₁₋₆ alkyl group; X is —NR⁴—, —NR⁴NR⁴—, or —S—; M has the formula: 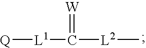
and the variables L¹, L², R¹, R², R³, Q, m, and n are selected from the respective groups of chemical moieties or integers later defined in the detailed description.

Particular embodiments of compounds of the invention include those having the formula: 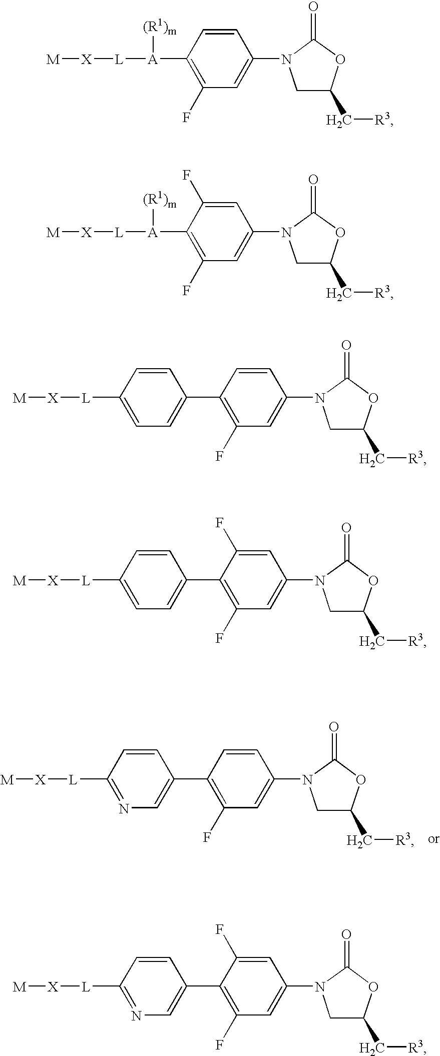
or a pharmaceutically acceptable salt, ester or prodrug thereof, wherein A, L, M, X, R¹, R³, and m are selected from the respective groups of chemical moieties or integers later defined in the detailed description.

In addition, the invention provides methods of synthesizing the foregoing compounds. Following synthesis, an effective amount of one or more of the compounds may be formulated with a pharmaceutically acceptable carrier for administration to a mammal for use as an anti-cancer, anti-microbial, anti-biotic, anti-fungal, anti-parasitic or anti-viral agent, or to treat a proliferative disease, an inflammatory disease or a gastrointestinal motility disorder. The compounds or formulations may be administered, for example, via oral, parenteral, or topical routes, to provide an effective amount of the compound to the mammal.

The foregoing and other aspects and embodiments of the invention may be more fully understood by reference to the following detailed description and claims.

### DETAILED DESCRIPTION OF THE INVENTION

The present invention provides a family of compounds that can be used as anti-proliferative agents and/or anti-infective agents. The compounds may be used without limitation, for example, as anti-cancer, anti-microbial, anti-bacterial, anti-fungal, anti-parasitic and/or anti-viral agents. Further, the present invention provides a family of compounds that can be used without limitation as anti-inflammatory agents, for example, for use in treating chronic inflammatory airway diseases, and/or as prokinetic agents, for example, for use in treating gastrointestinal motility disorders such as gastroesophageal reflux disease, gastroparesis (diabetic and post surgical), irritable bowel syndrome, and constipation.

### 1. DEFINITIONS

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties. Ring double bonds, as used herein, are double bonds that are formed between two adjacent ring atoms (e.g., C═C, C═N, or N═N).

The present invention is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

The compounds described herein may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic, and geometric isomeric forms of a structure are intended, unless the specific stereochemistry or isomeric form is specifically indicated. All processes used to prepare compounds of the present invention and intermediates made therein are considered to be part of the present invention. All tautomers of shown or described compounds are also considered to be part of the present invention.

When any variable (e.g., R¹) occurs more than one time in any constituent or formula for a compound, its definition at each occurrence is independent of its definition at every other occurrence. Thus, for example, if a group is shown to be substituted with 0-2 R¹ moieties, then the group may optionally be substituted with up to two R¹ moieties and R¹ at each occurrence is selected independently from the definition of R¹. Also, combinations of substituents and/or variables are permissible, but only if such combinations result in stable compounds.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such substituent. Combinations of substituents and/or variables are permissible, but only if such combinations result in stable compounds.

Compounds of the present invention that contain nitrogens can be converted to N-oxides by treatment with an oxidizing agent (e.g., 3-chloroperoxybenzoic acid (m-CPBA) and/or hydrogen peroxides) to afford other compounds of the present invention. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds of the present invention can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁₋₆ alkyl, C₁₋₆ alkenyl, C₁₋₆ alkynyl, C₃₋₁₄ carbocycle, or 3-14-membered heterocycle) derivatives.

When an atom or chemical moiety is followed by a subscripted numeric range (e.g., C₁₋₆), the invention is meant to encompass each number within the range as well as all intermediate ranges. For example, “C₁₋₆ alkyl” is meant to include alkyl groups with 1, 2, 3, 4, 5, 6, 1-6, 1-5, 1-4, 1-3, 1-2, 2-6, 2-5, 2-4, 2-3, 3-6, 3-5, 3-4, 4-6, 4-5, and 5-6 carbons.

As used herein, “alkyl” is intended to include both branched and straight-chain saturated aliphatic hydrocarbon groups having the specified number of carbon atoms. For example, C₁₋₆ alkyl is intended to include C₁, C₂, C₃, C₄, C₅, and C₆ alkyl groups. Examples of alkyl include, but are not limited to, methyl, ethyl, n-propyl, i-propyl, n-butyl, s-butyl, t-butyl, n-pentyl, s-pentyl, and n-hexyl.

As used herein, “alkenyl” is intended to include hydrocarbon chains of either straight or branched configuration having one or more carbon-carbon double bonds occurring at any stable point along the chain. For example, C₂₋₆ alkenyl is intended to include C₂, C₃, C₄, C₅, and C₆ alkenyl groups. Examples of alkenyl include, but are not limited to, ethenyl and propenyl.

As used herein, “alkynyl” is intended to include hydrocarbon chains of either straight or branched configuration having one or more carbon-carbon triple bonds occurring at any stable point along the chain. For example, C₂₋₆ alkynyl is intended to include C₂, C₃, C₄, C₅, and C₆ alkynyl groups. Examples of alkynyl include, but are not limited to, ethynyl and propynyl.

As used herein, “halo” or “halogen” refers to fluoro, chloro, bromo, and iodo. “Counterion” is used to represent a small, negatively charged species such as chloride, bromide, hydroxide, acetate, and sulfate.

As used herein, “carbocycle” or “carbocyclic ring” is intended to mean any stable monocyclic, bicyclic, or tricyclic ring having the specified number of carbons, any of which may be saturated, unsaturated, or aromatic. For example a C₃₋₁₄ carbocycle is intended to mean a mono-, bi-, or tricyclic ring having 3, 4, 5, 6, 7, 8, 9, 10, 11, 12, 13, or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl, and tetrahydronaphthyl. Bridged rings are also included in the definition of carbocycle, including, for example, [3.3.0]bicyclooctane, [4.3.0]bicyclononane, [4.4.0]bicyclodecane, and [2.2.2]bicyclooctane. A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge. Fused (e.g., naphthyl and tetrahydronaphthyl) and spiro rings are also included.

As used herein, the term “heterocycle” or “heterocyclic” is intended to mean any stable monocyclic, bicyclic, or tricyclic ring which is saturated, unsaturated, or aromatic and comprises carbon atoms and one or more ring heteroatoms, e.g., 1 or 1-2 or 1-3 or 1-4 or 1-5 or 1-6 heteroatoms, independently selected from the group consisting of nitrogen, oxygen, and sulfur. A bicyclic or tricyclic heterocycle may have one or more heteroatoms located in one ring, or the heteroatoms may be located in more than one ring. The nitrogen and sulfur heteroatoms may optionally be oxidized (i.e., N→O and S(O)ₚ, where p=1 or 2). When a nitrogen atom is included in the ring it is either N or NH, depending on whether or not it is attached to a double bond in the ring (i.e., a hydrogen is present if needed to maintain the tri-valency of the nitrogen atom). The nitrogen atom may be substituted or unsubstituted (i.e., N or NR wherein R is H or another substituent, as defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. Bridged rings are also included in the definition of heterocycle. A bridged ring occurs when one or more atoms (i.e., C, O, N, or S) link two non-adjacent carbon or nitrogen atoms. Preferred bridges include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge. Spiro and fused rings are also included.

As used herein, the term “aromatic heterocycle” or “heteroaryl” is intended to mean a stable 5, 6, or 7-membered monocyclic or bicyclic aromatic heterocyclic ring or 7, 8, 9, 10, 11, or 12-membered bicyclic aromatic heterocyclic ring which consists of carbon atoms and one or more heteroatoms, e.g., 1 or 1-2 or 1-3 or 1-4 or 1-5 or 1-6 heteroatoms, independently selected from the group consisting of nitrogen, oxygen, and sulfur. In the case of bicyclic heterocyclic aromatic rings, only one of the two rings needs to be aromatic (e.g., 2,3-dihydroindole), though both may be (e.g., quinoline). The second ring can also be fused or bridged as defined above for heterocycles. The nitrogen atom may be substituted or unsubstituted (i.e., N or NR wherein R is H or another substituent, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heterocycles include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluene sulfonic.

The pharmaceutically acceptable salts of the present invention can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed. (Mack Publishing Company, 1990).

Since prodrugs are known to enhance numerous desirable qualities of pharmaceuticals (e.g., solubility, bioavailability, manufacturing, etc.) the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a mammalian subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug of the present invention is administered to a mammalian subject, cleaves to form a free hydroxyl, free amino, or free sulfhydryl group, respectively. Examples of prodrugs include, but are not limited to, acetate, formate, and benzoate derivatives of alcohol and amine functional groups in the compounds of the present invention.

“Stable compound” and “stable structure” are meant to indicate a compound that is sufficiently robust to survive isolation to a useful degree of purity from a reaction mixture, and formulation into an efficacious therapeutic agent.

As used herein, “treating” or “treatment” means the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

As used herein, “mammal” refers to human and non-human patients.

As used herein, the term “effective amount” refers to an amount of a compound, or a combination of compounds, of the present invention effective when administered alone or in combination as an anti-proliferative and/or anti-infective agent. The combination of compounds is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased anti-proliferative and/or anti-infective effect, or some other beneficial effect of the combination compared with the individual components.

All percentages and ratios used herein, unless otherwise indicated, are by weight.

Throughout the description, where compositions are described as having, including, or comprising specific components, it is contemplated that compositions also consist essentially of, or consist of, the recited components. Similarly, where processes are described as having, including, or comprising specific process steps, the processes also consist essentially of, or consist of, the recited processing steps. Further, it should be understood that the order of steps or order for performing certain actions are immaterial so long as the invention remains operable. Moreover, two or more steps or actions may be conducted simultaneously.

### 2. COMPOUNDS OF THE INVENTION

In one aspect, the invention provides compounds having the formula: 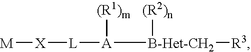
or a pharmaceutically acceptable salt, ester or prodrug thereof, wherein:

A is selected from the group consisting of:
phenyl, pyridyl, pyrazinyl, pyrimidinyl, and pyridazinyl;

B is selected from the group consisting of:
phenyl, pyridyl, pyrazinyl, pyrimidinyl, and pyridazinyl;

Het-CH₂—R³ is selected from the group consisting of:

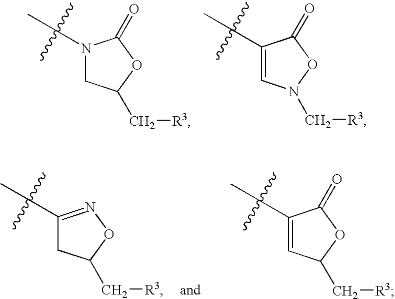

M has the formula: 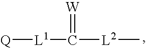
wherein
   L¹ is a bond or C₁₋₆ alkyl optionally substituted with one or more R⁴ groups;
   L² is a bond or C₁₋₆ alkyl optionally substituted with one or more R⁴ groups;
   Q is selected from the group consisting of:
      a) H, b) —NR⁴R⁴, c) —OR⁴, and d) C₁₋₆ alkyl optionally substituted with one or more R⁴ groups; and
   W is selected from the group consisting of O and S;

X is selected from the group consisting of:
a) —NR⁴—, b) —NR⁴NR⁴—, and c) —S—;

L is C₁₋₆ alkyl optionally substituted with one or more R⁴ groups;

R¹, at each occurrence, independently is selected from the group consisting of:
a) F, b) Cl, c) Br, d) I, e) —CF₃, f) —OR⁷, g) —CN, h) —NO₂, i) —NR⁷R⁷, j) —C(O)R⁷, k) —C(O)OR⁷, l) —OC(O)R⁷, m) —C(O)NR⁷R⁷, n) —NR⁷C(O)R⁷, o) —OC(O)NR⁷R⁷, p) —NR⁷C(O)OR⁷, q) —NR⁷C(O)NR⁷R⁷, r) —C(S)R⁷, s) —C(S)OR⁷, t) —OC(S)R⁷, u) —C(S)NR⁷R⁷, v) —NR⁷C(S)R⁷, w) —OC(S)NR⁷R⁷, x) —NR⁷C(S)OR⁷, y) —NR⁷C(S)NR⁷R⁷, z) —C(NR⁷)R⁷, aa) —C(NR⁷)OR⁷, bb) —OC(NR⁷)R⁷, cc) —C(NR⁷)NR⁷R⁷, dd) —NR⁷C(NR⁷)R⁷, ee) —OC(NR⁷)NR⁷R⁷, ff) —NR⁷C(NR⁷)OR⁷, gg) —NR⁷C(NR⁷)NR⁷R⁷, hh) —S(O)ₚR⁷, ii) —SO₂NR⁷R⁷, and jj) R⁷;

R², at each occurrence, independently is selected from the group consisting of:
a) F, b) Cl, c) Br, d) I, e) —CF₃, f) —OR⁷, g) —CN, h) —NO₂, i) —NR⁷R⁷, j) —C(O)R⁷, k) —C(O)OR⁷, l) —OC(O)R⁷, m) —C(O)NR⁷R⁷, n) —NR⁷C(O)R⁷, o) —OC(O)NR⁷R⁷, p) —NR⁷C(O)OR⁷, q) —NR⁷C(O)NR⁷R⁷, r) —C(S)R⁷, s) —C(S)OR⁷, t) —OC(S)R⁷, u) —C(S)NR⁷R⁷, v) —NR⁷C(S)R⁷, w) —OC(S)NR⁷R⁷, x) —NR⁷C(S)OR⁷, y) —NR⁷C(S)NR⁷R⁷, z) —C(NR⁷)R⁷, aa) —C(NR⁷)OR⁷, bb) —OC(NR⁷)R⁷, cc) —C(NR⁷)NR⁷R⁷, dd) —NR⁷C(NR⁷)R⁷, ee) —OC(NR⁷)NR⁷R⁷, ff) —NR⁷C(NR⁷)OR⁷, gg) —NR⁷C(NR⁷)NR⁷R⁷, hh) —S(O)ₚR⁷, ii)-SO₂NR⁷R⁷, and jj) R⁷;

R³ is selected from the group consisting of:
a) —OR⁷, b) —NR⁷R⁷, c) —C(O)R⁷, d) —C(O)OR⁷, e) —OC(O)R⁷, f) —C(O)NR⁷R⁷, g) —NR⁷C(O)R⁷, h) —OC(O)NR⁷R⁷, i) —NR⁷C(O)OR⁷, j) —NR⁷C(O)NR⁷R⁷, k) —C(S)R⁷, l) —C(S)OR⁷, m) —OC(S)R⁷, n) —C(S)NR⁷R⁷, o) —NR⁷C(S)R⁷, p) —OC(S)NR⁷R⁷, q) —NR⁷C(S)OR⁷, r) —NR⁷C(S)NR⁷R⁷, s) —C(NR⁷)R⁷, t) —C(NR⁷)OR⁷, u) —OC(NR⁷)R⁷, v) —C(NR⁷)NR⁷R⁷, w) —NR⁷C(NR⁷)R⁷, x) —OC(NR⁷)NR⁷R⁷, y) —NR⁷C(NR⁷)OR⁷, z) —NR⁷C(NR⁷)NR⁷R⁷, aa) —S(O)ₚR⁷, bb) —SO₂NR⁷R⁷, and cc) R⁷;

R⁴, at each occurrence, independently is selected from the group consisting of:
a) H, b) ═O, c) ═S, d) ═NR⁵, e) ═NOR⁵, f) ═N—NR⁵R⁵, g) —OR⁵, h) —NO₂, i) —NR⁵R⁵, j) —C(O)R⁵, k) —C(O)OR⁵, l) —OC(O)R⁵, m) —C(O)NR⁵R⁵, n) —NR⁵C(O)R⁵, o) —OC(O)NR⁵R⁵, p) —NR⁵C(O)OR⁵, q) —NR⁵C(O)NR⁵R⁵, r) —C(S)R⁵, s) —C(S)OR⁵, t) —OC(S)R⁵, u) —C(S)NR⁵R⁵, v) —NR⁵C(S)R⁵, w) —OC(S)NR⁵R⁵, x) —NR⁵C(S)OR⁵, y) —NR⁵C(S)NR⁵R⁵, z) —C(NR⁵)R⁵, aa) —C(S)OR⁵, bb) —OC(NR⁵)R⁵, cc) —C(NR⁵)NR⁵R⁵, dd) —NR⁵C(NR⁵)R⁵, ee) —OC(NR⁵)NR⁵R⁵, ff) —NR⁵C(NR⁵)OR⁵, gg) —NR⁵C(NR⁵)NR⁵R⁵, hh) —S(O)ₚR⁵, and ii) R⁵;

R⁵, at each occurrence, independently is selected from the group consisting of:
a) H, b) C₁₋₆ alkyl, c) —C(O)—C₁₋₆ alkyl, and d) —C(O)O-C₁₋₆ alkyl, wherein any of b)-d) optionally is substituted with one or more R⁶ groups;

R⁶, at each occurrence, independently is selected from the group consisting of:
a) —OH, b) —OC₁₋₆ alkyl, c) —SH, d) —NO₂, e) —NH₂, f) —NHC₁₋₆ alkyl, g) —N(C₁₋₆ alkyl)₂, h) —C(O)H, i) —C(O)OH, j) —C(O)C₁₋₆ alkyl, k) —OC(O)C₁₋₆ alkyl, l) —C(O)OC₁₋₆ alkyl, m) —C(O)NH₂, n) —C(O)NHC₁₋₆ alkyl, o) —C(O)N(C₁₋₆ alkyl)₂, p) —NHC(O)C₁₋₆ alkyl, and q) —S(O)ₚC₁— alkyl;

R⁷, at each occurrence, independently is selected from the group consisting of:
a) H, b) C₁₋₆ allyl, c) C₂₋₆ alkenyl, d) C₂₋₆ alkynyl, e) C₃₋₁₄ saturated, unsaturated, or aromatic carbocycle, f) 3-14 membered saturated, unsaturated, or aromatic heterocycle comprising one or more heteroatoms selected from the group consisting of nitrogen, oxygen, and sulfur, g) —C(O)—C₁₋₆ alkyl, h) —C(O)—C₂₋₆ alkenyl, i) —C(O)—C₂₋₆ alkynyl, j) —C(O)—C₃₋₁₄ saturated, unsaturated, or aromatic carbocycle, k) —C(O)-3-14 membered saturated, unsaturated, or aromatic heterocycle comprising one or more heteroatoms selected from the group consisting of nitrogen, oxygen, and sulfur, l) —C(O)O-C₁₋₆ alkyl, m) —C(O)O—C₂₋₆ alkenyl, n) —C(O)O—C₂₋₆ alkynyl, o) —C(O)O—C₃₋₁₄ saturated, unsaturated, or aromatic carbocycle, and p) —C(O)O-3-14 membered saturated, unsaturated, or aromatic heterocycle comprising one or more heteroatoms selected from the group consisting of nitrogen, oxygen, and sulfur, wherein any of b)-p) optionally is substituted with one or more R⁸ groups;

R⁸, at each occurrence, is independently selected from the group consisting of:
a) F, b) Cl, c) Br, d) I, e) ═O, f) ═S, g) ═NR⁹, h) ═NOR⁹, i) ═N—NR⁹R⁹, j) —CF₃, k) OR⁹, l)—CN, m) —NO₂, n) —NR⁹R⁹, o) —C(O)R⁹, p) —C(O)OR⁹, q) —OC(O)R⁹, r) —C(O)NR⁹R⁹, s) —NR⁹C(O)R⁹, t) —OC(O)NR⁹R⁹, u) —NR⁹C(O)OR⁹, v) —NR⁹C(O)NR⁹R⁹, w) —C(S)R⁹, x) —C(S)OR⁹, y) —OC(S)R⁹, z) —C(S)NR⁹R⁹, aa) —NR⁹C(S)R⁹, bb) —OC(S)NR⁹R⁹, cc) —NR⁹C(S)OR⁹, dd) —NR⁹C(S)NR⁹R⁹, ee) —C(NR⁹)R⁹, ff) —C(NR⁹)OR⁹, gg) —OC(NR⁹)R⁹, hh) —C(NR⁹)NR⁹R⁹, ii) —NR⁹C(NR⁹)R⁹, jj) —OC(NR⁹)NR⁹R⁹, kk) —NR⁹C(NR⁹)OR⁹, ll) —NR⁹C(NR⁹)NR⁹R⁹, m) —S(O)ₚR⁹, m) —SO₂NR⁹R⁹, and oo) R⁹;

R⁹, at each occurrence, independently is selected from the group consisting of:
a) H, b) C₁₋₆ alkyl, c) C₂₋₆ alkenyl, d) C₂₋₆ alkynyl, e) C₃₋₁₄ saturated, unsaturated, or aromatic carbocycle, f) 3-14 membered saturated, unsaturated, or aromatic heterocycle comprising one or more heteroatoms selected from the group consisting of nitrogen, oxygen, and sulfur, g) —C(O)—C₁₋₆ alkyl, h) —C(O)—C₂₋₆ alkenyl, i) C(O)—C₂₋₆ alkynyl, j) —C(O)—C₃₋₁₄ saturated, unsaturated, or aromatic carbocycle, k) —C(O)-3-14 membered saturated, unsaturated, or aromatic heterocycle comprising one or more heteroatoms selected from the group consisting of nitrogen, oxygen, and sulfur, l) —C(O)O—C₁₋₆ alkyl, m) —C(O)O—C₂₋₆ alkenyl, n) —C(O)O—C₂₋₆ alkynyl, o) —C(O)O—C₃₋₁₄ saturated, unsaturated, or aromatic carbocycle, and p) —C(O)O-3-14 membered saturated, unsaturated, or aromatic heterocycle comprising one or more heteroatoms selected from the group consisting of nitrogen, oxygen, and sulfur,
   wherein any of b)-p) optionally is substituted with one or more moieties selected from the group consisting of:
      a) F, b) Cl, c) Br, d) I, e) —CF₃, f) —OH, g) —OC₁₋₆ alkyl, h) —SH, i) —SC₁₋₆ alkyl, j) —CN, k) —NO₂, l) —NH₂, m) —NHC— alkyl, n) —N(C₁₋₆ alkyl)₂, o) —C(O)C₁₋₆ alkyl, p) —OC(O)C₁₋₆ alkyl, q)-C(O)OC₁₋₆ alkyl, r) —C(O)NH₂, s) —C(O)NHC₁₋₆ alkyl, t) —C(O)N(C₁₋₆ alkyl)₂, u) —NHC(O)C₁₋₆ alkyl, v) —SO₂NH₂—, w) —SO₂NHC₁₋₆ alkyl, x) —SO₂N(C₁₋₆ alkyl)₂, and y) —S(O)ₚC₁₋₆ alkyl;

m is 0, 1, 2, 3, or 4;

n is 0, 1, 2, 3, or 4; and

p, at each occurrence, independently is 0, 1, or 2,

and wherein the compound does not have the formula selected from the group consisting of:

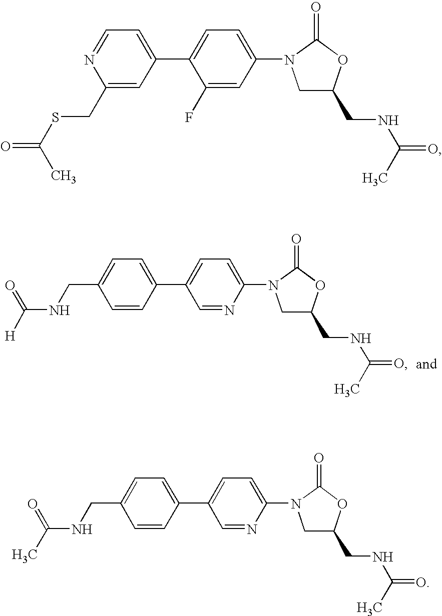

Particular embodiments of the invention include compounds having the formula: 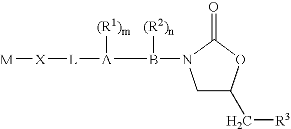
or a pharmaceutically acceptable salt, ester or prodrug thereof, wherein A, B, L, M, R¹, R², R³, X, m, and n are defined above.

Other embodiments include compounds having the formula: 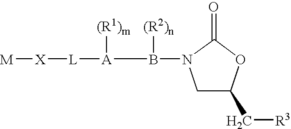
or a pharmaceutically acceptable salt, ester or prodrug thereof, wherein A, B, L, M, R¹, R², R³, X, m, and n are defined as described above.

Particular compounds include those where A is selected from the group consisting of phenyl and pyridyl; B is selected from the group consisting of phenyl and pyridyl; m is 0, 1, or 2;

and n is 0, 1, or 2.

In some embodiments, A-B is: 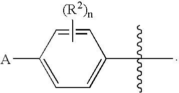
wherein A, R², and n are defined as described above. In particular embodiments, A-B is: 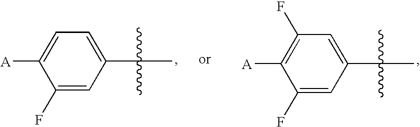
wherein A is defined as described above.

In various embodiments, A-B is: 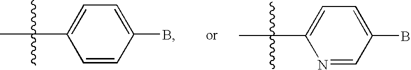
wherein B is defined as described in above.

In some embodiments, R³ is —NHC(O)R⁷. Particular compounds according to these embodiments include those where R⁷ is —CH₃. In other embodiments, R³ is

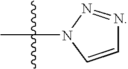

Particular embodiments of the invention include compounds having the formula: 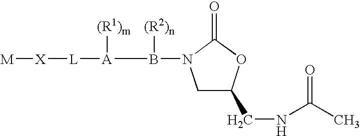
or a pharmaceutically acceptable salt, ester or prodrug thereof, wherein A, B, L, M, R¹, R², X, m, and n are defined as described above.

Other embodiments of the invention include compounds having the formula: 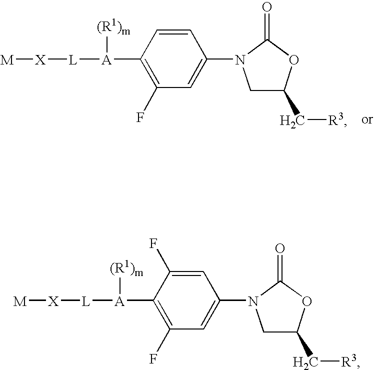
or a pharmaceutically acceptable salt, ester or prodrug thereof, wherein A, L, M, R¹, R³, X, and m are defined as described above. Particular compounds according to these embodiments include compounds having the formula: 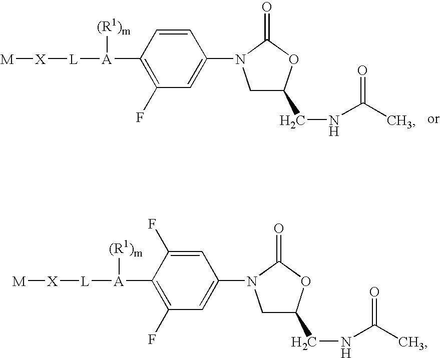
or a pharmaceutically acceptable salt, ester or prodrug thereof, wherein A, L, M, R¹, X, and m are defined as described above.

Some embodiments of the invention include compounds having the formula: 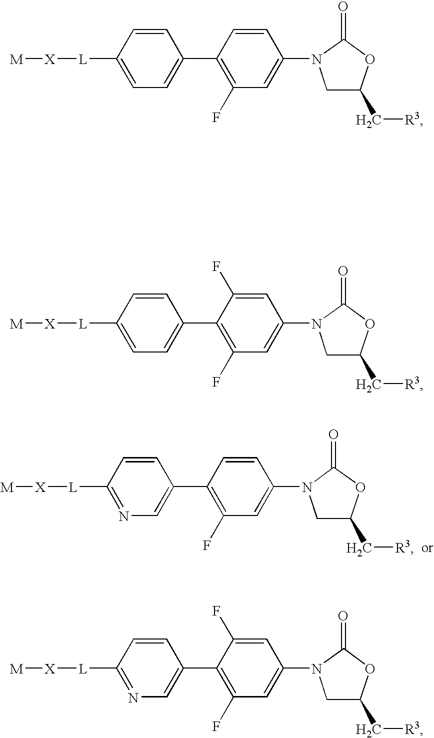
or a pharmaceutically acceptable salt, ester or prodrug thereof, wherein L, M, X, and R³ are defined as described above. Particular compounds according to these embodiments include those having the formula: 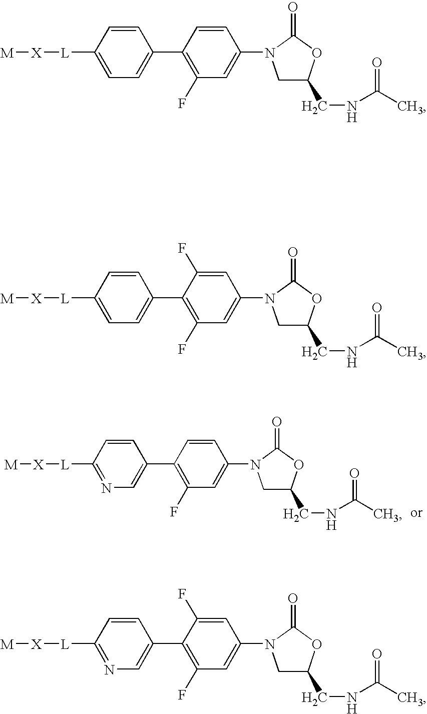
or a pharmaceutically acceptable salt, ester or prodrug thereof, wherein L, M, and X are defined as described above.

In some embodiments, M is: 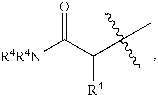
wherein R⁴, at each occurrence, independently is defined as described in claim 1. Particular compounds according to these embodiments include those having the formula:

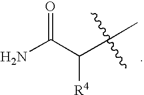

In other embodiments, M is: 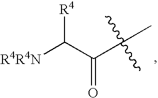
wherein R⁴, at each occurrence, independently is defined as described in claim 1. Particular compounds according to these embodiments include those having the formula:

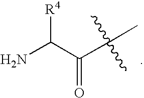

In certain embodiments, X is —NH—. In other embodiments, X is:

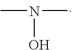

In another aspect, the invention provides methods for synthesizing compounds according to the invention. In yet another aspect, the invention provides a pharmaceutical composition comprising an effective amount of one or more of the foregoing compounds and a pharmaceutically acceptable carrier. Suitable formulating agents are described in detail in section 4 hereinbelow.

One or more of the foregoing compounds may also be incorporated into a medical device. For example, a medical device, such as a medical stent, can contain or be coated with one or more of the compounds of the invention.

In another aspect, the invention provides a method for treating a microbial infection, a fungal infection, a viral infection, a parasitic disease, a proliferative disease, an inflammatory disease, or a gastrointestinal motility disorder in a mammal. The method involves administering an effective amount of one or more compounds or pharmaceutical compositions of the invention, for example, via oral, parenteral or topical routes.

The invention provides a method of treating a disorder in a mammal comprising the step of administering to the mammal an effective amount of one or more compounds of the invention thereby to ameliorate a symptom of a particular disorder. Such a disorder can be selected from the group consisting of a skin infection, nosocomial pneumonia, post-viral pneumonia, an abdominal infection, a urinary tract infection, bacteremia, septicemia, endocarditis, an atrio-ventricular shunt infection, a vascular access infection, meningitis, surgical prophylaxis, a peritoneal infection, a bone infection, a joint infection, a methicillin-resistant Staphylococcus aureus infection, a vancomycin-resistant Enterococci infection, a linezolid-resistant organism infection, and tuberculosis.

### 3. CHARACTERIZATION OF COMPOUNDS OF THE INVENTION

Compounds designed, selected and/or optimized by methods described above, once produced, may be characterized using a variety of assays known to those skilled in the art to determine whether the compounds have biological activity. For example, the molecules may be characterized by conventional assays, including but not limited to those assays described below, to determine whether they have a predicted activity, binding activity and/or binding specificity.

Furthermore, high-throughput screening may be used to speed up analysis using such assays. As a result, it may be possible to rapidly screen the molecules described herein for activity, for example, as anti-cancer, anti-bacterial, anti-fungal, anti-parasitic or anti-viral agents. Also, it may be possible to assay how the compounds interact with a ribosome or ribosomal subunit and/or are effective as modulators (for example, inhibitors) of protein synthesis using techniques known in the art. General methodologies for performing high-throughput screening are described, for example, in Devlin, High Throughput Screening, (Marcel Dekker, 1998); and U.S. Pat. No. 5,763,263. High-throughput assays can use one or more different assay techniques including, but not limited to, those described below.

(1) Surface Binding Studies. A variety of binding assays may be useful in screening new molecules for their binding activity. One approach includes surface plasmon resonance (SPR) that can be used to evaluate the binding properties of molecules of interest with respect to a ribosome, ribosomal subunit or a fragment thereof.

SPR methodologies measure the interaction between two or more macromolecules in real-time through the generation of a quantum-mechanical surface plasmon. One device, (BIAcore Biosensor® from Pharmacia Biosensor, Piscatawy, N.J.) provides a focused beam of polychromatic light to the interface between a gold film (provided as a disposable biosensor “chip”) and a buffer compartment that can be regulated by the user. A 100 nm thick “hydrogel” composed of carboxylated dextran that provides a matrix for the covalent immobilization of analytes of interest is attached to the gold film. When the focused light interacts with the free electron cloud of the gold film, plasmon resonance is enhanced. The resulting reflected light is spectrally depleted in wavelengths that optimally evolved the resonance. By separating the reflected polychromatic light into its component wavelengths (by means of a prism), and determining the frequencies that are depleted, the BIAcore establishes an optical interface which accurately reports the behavior of the generated surface plasmon resonance. When designed as above, the plasmon resonance (and thus the depletion spectrum) is sensitive to mass in the evanescent field (which corresponds roughly to the thickness of the hydrogel). If one component of an interacting pair is immobilized to the hydrogel, and the interacting partner is provided through the buffer compartment, the interaction between the two components can be measured in real time based on the accumulation of mass in the evanescent field and its corresponding effects of the plasmon resonance as measured by the depletion spectrum. This system permits rapid and sensitive real-time measurement of the molecular interactions without the need to label either component.

(2) Fluorescence Polarization. Fluorescence polarization (FP) is a measurement technique that can readily be applied to protein-protein, protein-ligand, or RNA-ligand interactions in order to derive IC₅₀s and Kds of the association reaction between two molecules. In this technique one of the molecules of interest is conjugated with a fluorophore. This is generally the smaller molecule in the system (in this case, the compound of interest). The sample mixture, containing both the ligand-probe conjugate and the ribosome, ribosomal subunit or fragment thereof, is excited with vertically polarized light. Light is absorbed by the probe fluorophores, and re-emitted a short time later. The degree of polarization of the emitted light is measured. Polarization of the emitted light is dependent on several factors, but most importantly on viscosity of the solution and on the apparent molecular weight of the fluorophore. With proper controls, changes in the degree of polarization of the emitted light depends only on changes in the apparent molecular weight of the fluorophore, which in-turn depends on whether the probe-ligand conjugate is free in solution, or is bound to a receptor. Binding assays based on FP have a number of important advantages, including the measurement of IC₅₀s and Kds under true homogenous equilibrium conditions, speed of analysis and amenity to automation, and ability to screen in cloudy suspensions and colored solutions.

(3) Protein Synthesis. It is contemplated that, in addition to characterization by the foregoing biochemical assays, the compound of interest may also be characterized as a modulator (for example, an inhibitor of protein synthesis) of the functional activity of the ribosome or ribosomal subunit.

Furthermore, more specific protein synthesis inhibition assays may be performed by administering the compound to a whole organism, tissue, organ, organelle, cell, a cellular or subcellular extract, or a purified ribosome preparation and observing its pharmacological and inhibitory properties by determining, for example, its inhibition constant (IC₅₀) for inhibiting protein synthesis. Incorporation of ³H leucine or ³⁵S methionine, or similar experiments can be performed to investigate protein synthesis activity. A change in the amount or the rate of protein synthesis in the cell in the presence of a molecule of interest indicates that the molecule is a modulator of protein synthesis. A decrease in the rate or the amount of protein synthesis indicates that the molecule is a inhibitor of protein synthesis.

Furthermore, the compounds may be assayed for anti-proliferative or anti-infective properties on a cellular level. For example, where the target organism is a microorganism, the activity of compounds of interest may be assayed by growing the microorganisms of interest in media either containing or lacking the compound. Growth inhibition may be indicative that the molecule may be acting as a protein synthesis inhibitor. More specifically, the activity of the compounds of interest against bacterial pathogens may be demonstrated by the ability of the compound to inhibit growth of defined strains of human pathogens. For this purpose, a panel of bacterial strains can be assembled to include a variety of target pathogenic species, some containing resistance mechanisms that have been characterized. Use of such a panel of organisms permits the determination of structure-activity relationships not only in regards to potency and spectrum, but also with a view to obviating resistance mechanisms. The assays may be performed in microtiter trays according to conventional methodologies as published by The National Committee for Clinical Laboratory Standards (NCCLS) guidelines (NCCLS. M7-A5-Methods for Dilution Antimicrobial Susceptibility Tests for Bacteria That Grow Aerobically; Approved Standard-Fifth Edition. NCCLS Document M100-S12/M7 (ISBN 1-56238-394-9)).

### 4. FORMULATION AND ADMINISTRATION

The compounds of the invention may be useful in the prevention or treatment of a variety of human or other animal disorders, including for example, bacterial infection, fungal infections, viral infections, parasitic diseases, and cancer. It is contemplated that, once identified, the active molecules of the invention may be incorporated into any suitable carrier prior to use. The dose of active molecule, mode of administration and use of suitable carrier will depend upon the intended recipient and target organism. The formulations, both for veterinary and for human medical use, of compounds according to the present invention typically include such compounds in association with a pharmaceutically acceptable carrier.

The carrier(s) should be “acceptable” in the sense of being compatible with the other ingredients of the formulations and not deleterious to the recipient. Pharmaceutically acceptable carriers, in this regard, are intended to include any and all solvents, dispersion media, coatings, anti-bacterial and anti-fungal agents, isotonic and absorption delaying agents, and the like, compatible with pharmaceutical administration. The use of such media and agents for pharmaceutically active substances is known in the art. Except insofar as any conventional media or agent is incompatible with the active compound, use thereof in the compositions is contemplated. Supplementary active compounds (identified or designed according to the invention and/or known in the art) also can be incorporated into the compositions. The formulations may conveniently be presented in dosage unit form and may be prepared by any of the methods well known in the art of pharmacy/microbiology. In general, some formulations are prepared by bringing the compound into association with a liquid carrier or a finely divided solid carrier or both, and then, if necessary, shaping the product into the desired formulation.

A pharmaceutical composition of the invention should be formulated to be compatible with its intended route of administration. Examples of routes of administration include oral or parenteral, for example, intravenous, intradermal, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Useful solutions for oral or parenteral administration can be prepared by any of the methods well known in the pharmaceutical art, described, for example, in Remington's Pharmaceutical Sciences, 18th ed. (Mack Publishing Company, 1990). Formulations for parenteral administration can also include glycocholate for buccal administration, methoxysalicylate for rectal administration, or citric acid for vaginal administration. The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions which are solid at room temperature and liquid at body temperatures. Formulations also can include, for example, polyalkylene glycols such as polyethylene glycol, oils of vegetable origin, and hydrogenated naphthalenes. Formulations for direct administration can include glycerol and other compositions of high viscosity. Other potentially useful parenteral carriers for these drugs include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Formulations of the present invention suitable for oral administration may be in the form of: discrete units such as capsules, gelatin capsules, sachets, tablets, troches, or lozenges, each containing a predetermined amount of the drug; a powder or granular composition; a solution or a suspension in an aqueous liquid or non-aqueous liquid; or an oil-in-water emulsion or a water-in-oil emulsion. The drug may also be administered in the form of a bolus, electuary or paste. A tablet may be made by compressing or molding the drug optionally with one or more accessory ingredients. Compressed tablets may be prepared by compressing, in a suitable machine, the drug in a free-flowing form such as a powder or granules, optionally mixed by a binder, lubricant, inert diluent, surface active or dispersing agent. Molded tablets may be made by molding, in a suitable machine, a mixture of the powdered drug and suitable carrier moistened with an inert liquid diluent.

Oral compositions generally include an inert diluent or an edible carrier. For the purpose of oral therapeutic administration, the active compound can be incorporated with excipients. Oral compositions prepared using a fluid carrier for use as a mouthwash include the compound in the fluid carrier and are applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration may be in the form of a sterile aqueous preparation of the drug that may be in microcrystalline form, for example, in the form of an aqueous microcrystalline suspension. Liposomal formulations or biodegradable polymer systems may also be used to present the drug for both intra-articular and ophthalmic administration.

Formulations suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the drug with a dermatologically acceptable carrier such as a lotion, cream, ointment or soap. Particularly useful are carriers capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used.

For inhalation treatments, inhalation of powder (self-propelling or spray formulations) dispensed with a spray can, a nebulizer, or an atomizer can be used. Such formulations can be in the form of a fine powder for pulmonary administration from a powder inhalation device or self-propelling powder-dispensing formulations. In the case of self-propelling solution and spray formulations, the effect may be achieved either by choice of a valve having the desired spray characteristics (i.e., being capable of producing a spray having the desired particle size) or by incorporating the active ingredient as a suspended powder in controlled particle size. For administration by inhalation, the compounds also can be delivered in the form of an aerosol spray from pressured container or dispenser which contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents and bile salts. Transmucosal administration can be accomplished through the use of nasal sprays or suppositories. For transdermal administration, the active compounds typically are formulated into ointments, salves, gels, or creams as generally known in the art.

The active compounds may be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. Furthermore, administration can be by periodic injections of a bolus, or can be made more continuous by intravenous, intramuscular or intraperitoneal administration from an external reservoir (e.g., an intravenous bag).

Where adhesion to a tissue surface is desired the composition can include the drug dispersed in a fibrinogen-thrombin composition or other bioadhesive. The compound then can be painted, sprayed or otherwise applied to the desired tissue surface. Alternatively, the drugs can be formulated for parenteral or oral administration to humans or other mammals, for example, in effective amounts, e.g., amounts that provide appropriate concentrations of the drug to target tissue for a time sufficient to induce the desired effect.

Where the active compound is to be used as part of a transplant procedure, it can be provided to the living tissue or organ to be transplanted prior to removal of tissue or organ from the donor. The compound can be provided to the donor host. Alternatively or, in addition, once removed from the donor, the organ or living tissue can be placed in a preservation solution containing the active compound. In all cases, the active compound can be administered directly to the desired tissue, as by injection to the tissue, or it can be provided systemically, either by oral or parenteral administration, using any of the methods and formulations described herein and/or known in the art. Where the drug comprises part of a tissue or organ preservation solution, any commercially available preservation solution can be used to advantage. For example, useful solutions known in the art include Collins solution, Wisconsin solution, Belzer solution, Eurocollins solution and lactated Ringer's solution.

Active compound as identified or designed by the methods described herein can be administered to individuals to treat disorders (prophylactically or therapeutically). In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a drug as well as tailoring the dosage and/or therapeutic regimen of treatment with the drug.

In therapeutic use for treating, or combating, bacterial infections in mammals, the compounds or pharmaceutical compositions thereof will be administered orally, parenterally and/or topically at a dosage to obtain and maintain a concentration, that is, an amount, or blood-level or tissue level of active component in the animal undergoing treatment which will be anti-microbially effective. The term “effective amount” is understood to mean that the compound of the invention is present in or on the recipient in an amount sufficient to elicit biological activity, for example, anti-microbial activity, anti-fungal activity, anti-viral activity, anti-parasitic activity, and/or anti-proliferative activity. Generally, an effective amount of dosage of active component will be in the range of from about 0.1 to about 100, more preferably from about 1.0 to about 50 mg/kg of body weight/day. The amount administered will also likely depend on such variables as the type and extent of disease or indication to be treated, the overall health status of the particular patient, the relative biological efficacy of the compound delivered, the formulation of the drug, the presence and types of excipients in the formulation, and the route of administration.

Also, it is to be understood that the initial dosage administered may be increased beyond the above upper level in order to rapidly achieve the desired blood-level or tissue level, or the initial dosage may be smaller than the optimum and the daily dosage may be progressively increased during the course of treatment depending on the particular situation. If desired, the daily dose may also be divided into multiple doses for administration, for example, two to four times per day.

### 5. EXAMPLES

Nuclear magnetic resonance (NMR) spectra were obtained on a Bruker Avance 300 or Avance 500 spectrometer, or in some cases a GE-Nicolet 300 spectrometer. Common reaction solvents were either high performance liquid chromatography (HPLC) grade or American Chemical Society (ACS) grade, and anhydrous as obtained from the manufacturer unless otherwise noted. “Chromatography” or “purified by silica gel” refers to flash column chromatography using silica gel (EM Merck, Silica Gel 60, 230-400 mesh) unless otherwise noted. N-(t-Butoxycarbonyl)amino acid amides were synthesized according to literature procedures (e.g., Pozdnev, V. F. Tetrahedron Lett. 1995, 36 (39), 7115-7118).

Exemplary compounds in accordance with the invention are listed in Table 1.

TABLE 1

| Compound | |
|---|---|
| Number | Structure |
| 101 | 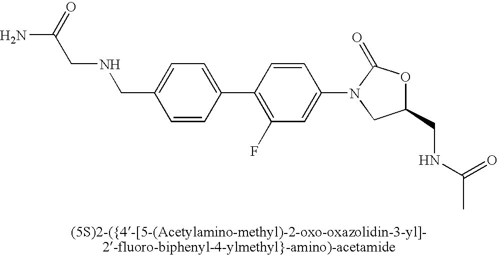 |
| 102 | 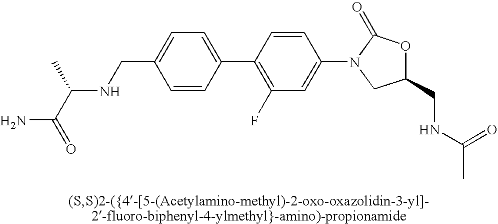 |
| 103 | 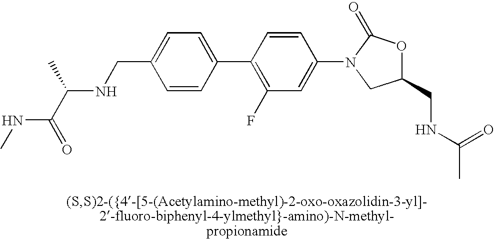 |
| 104 | 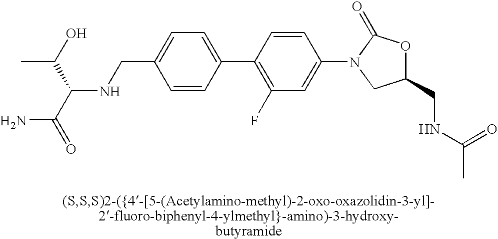 |
| 105 | 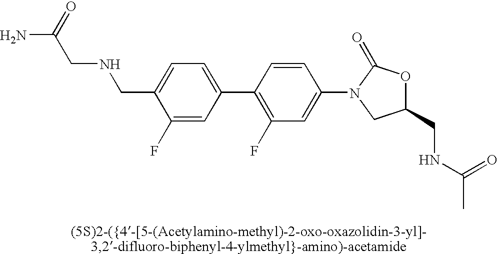 |
| 106 | 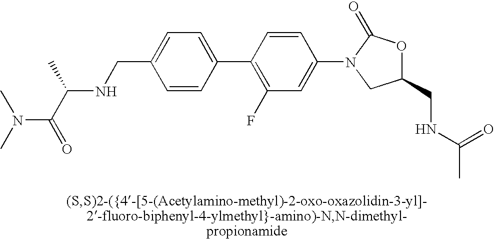 |
| 107 | 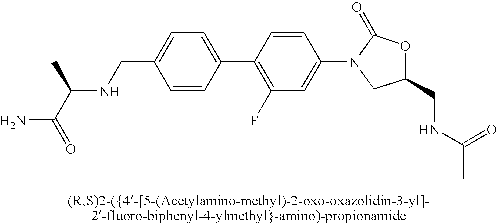 |
| 108 | 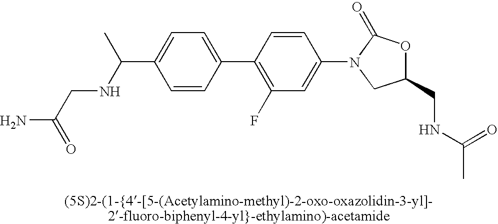 |
| 109 | 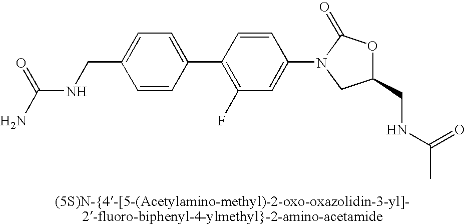 |
| 110 | 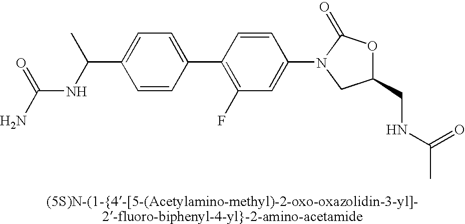 |
| 111 | 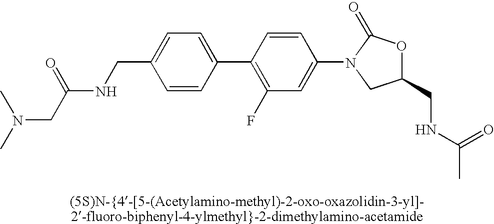 |
| 112 | 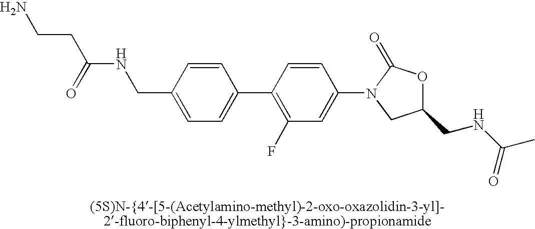 |
| 113 | 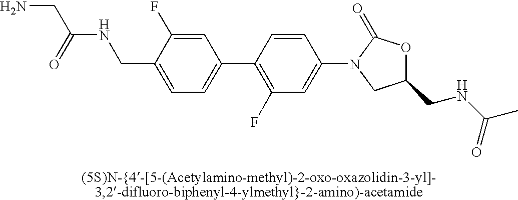 |
| 114 | 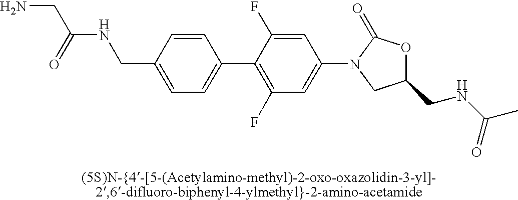 |
| 115 | 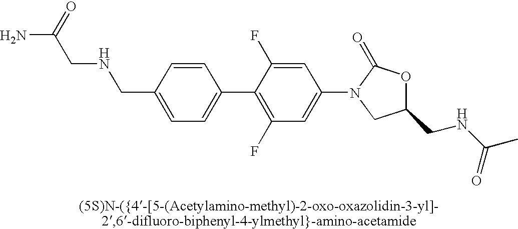 |
| 116 | 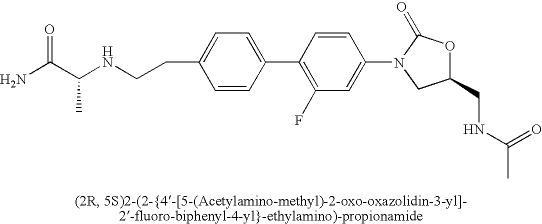 |
| 117 | 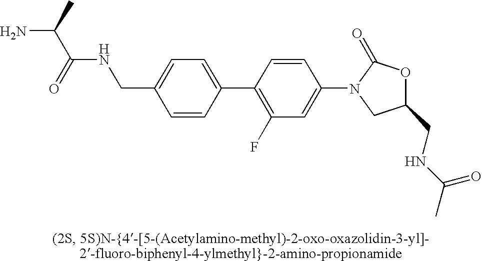 |
| 118 | 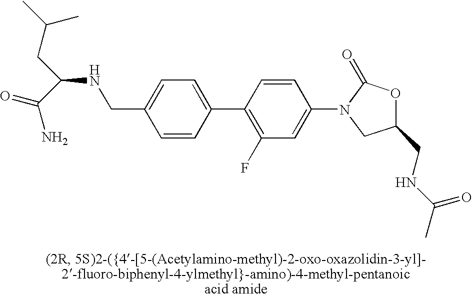 |
| 119 | 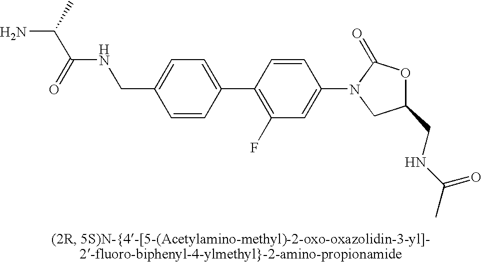 |
| 120 | 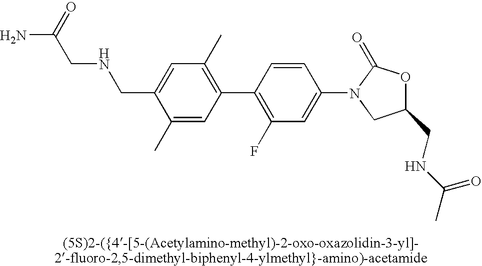 |
| 121 | 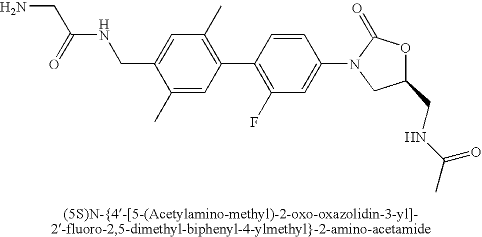 |
| 122 | 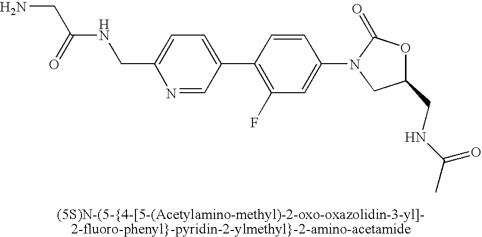 |
| 123 | 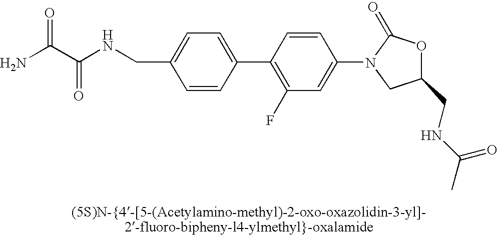 |
| 124 | 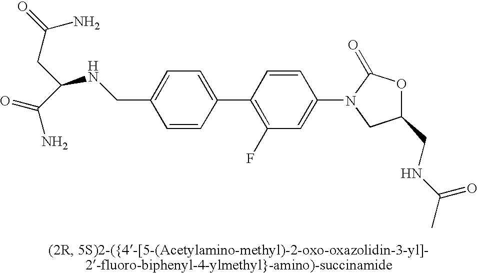 |
| 125 | 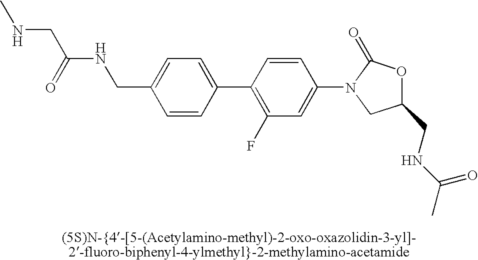 |
| 126 | 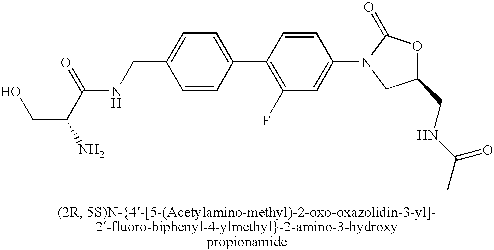 |
| 127 | 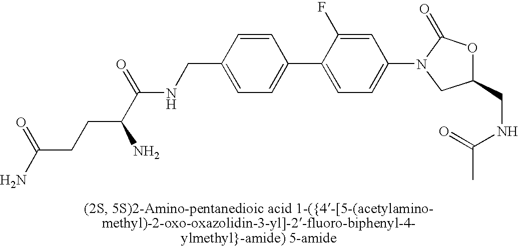 |
| 128 | 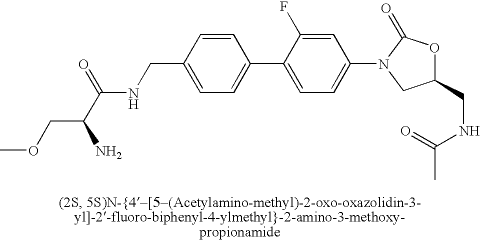 |
| 129 | 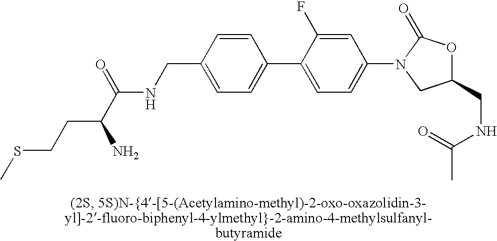 |
| 130 | 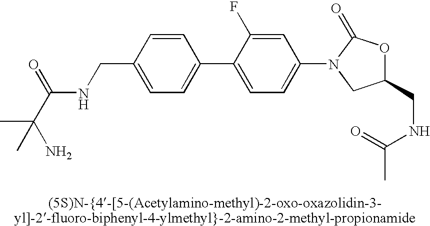 |
| 131 | 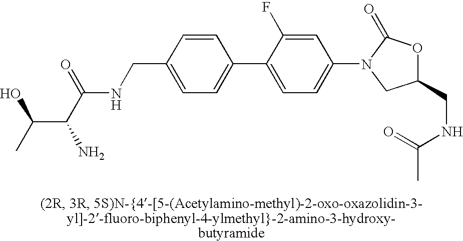 |
| 132 | 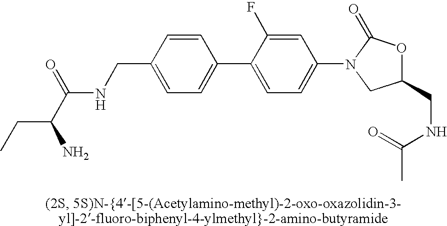 |
| 133 | 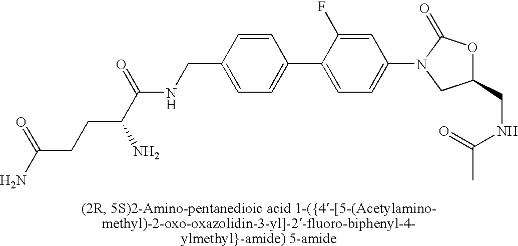 |
| 134 | 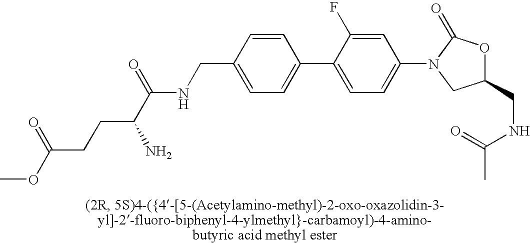 |
| 135 | 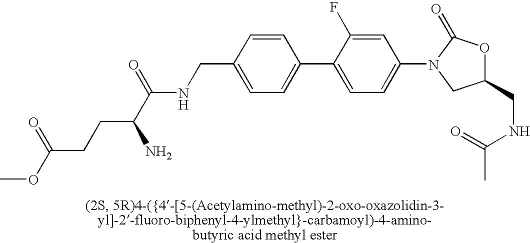 |
| 136 | 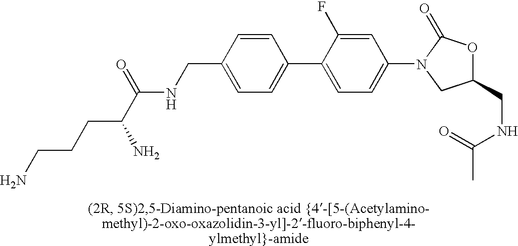 |
| 137 | 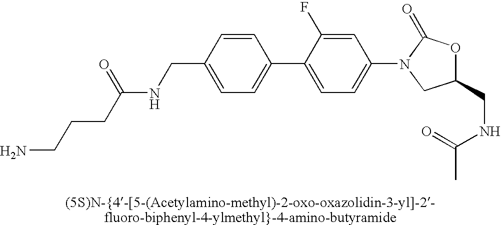 |
| 138 | 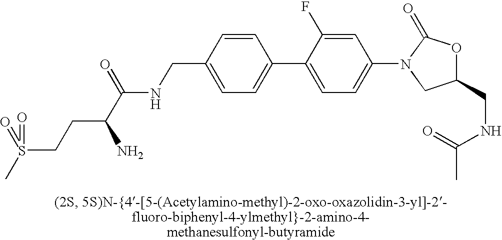 |
| 139 | 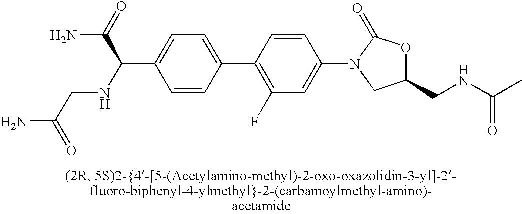 |
| 140 | 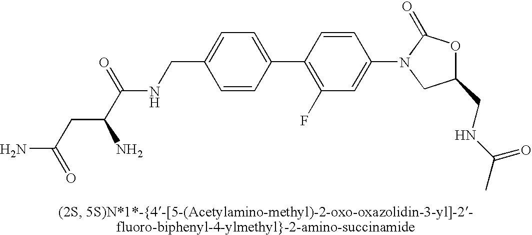 |
| 141 | 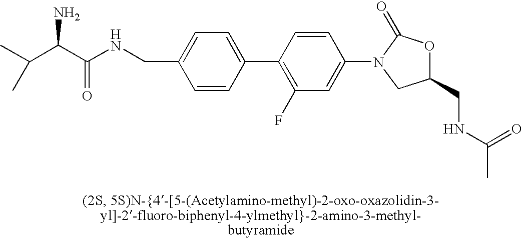 |
| 142 | 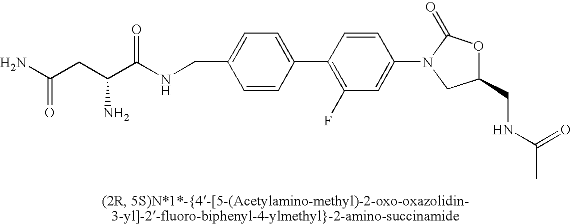 |
| 143 | 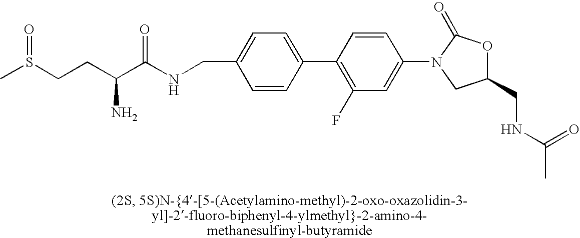 |
| 144 | 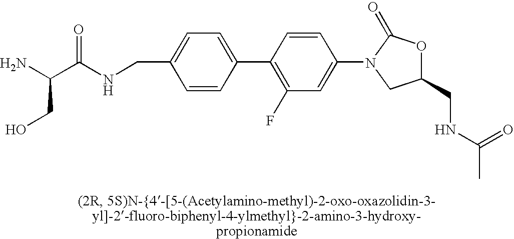 |
| 145 | 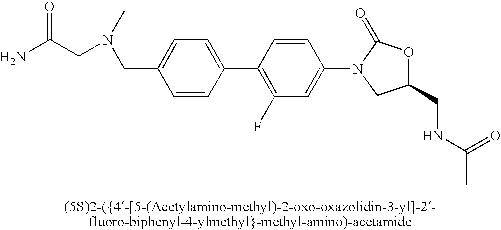 |
| 146 | 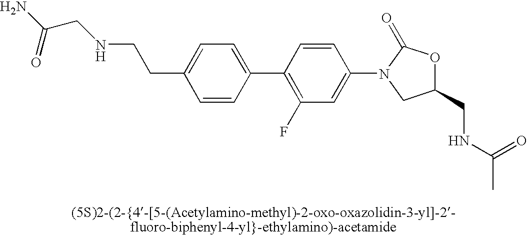 |
| 147 | 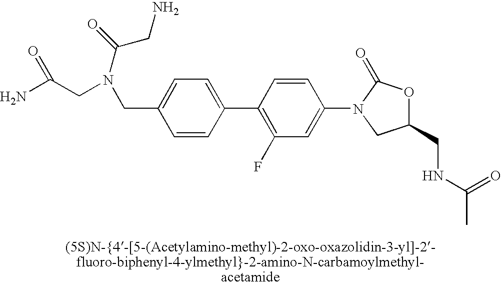 |
| 148 | 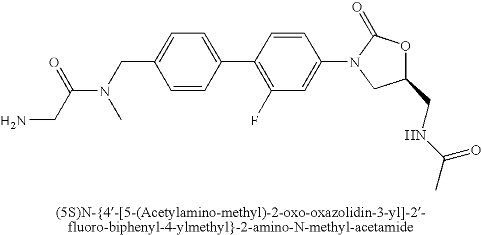 |
| 149 | 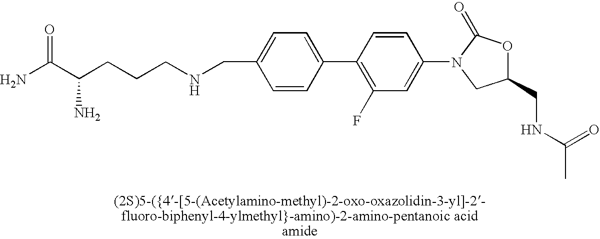 |
| 150 | 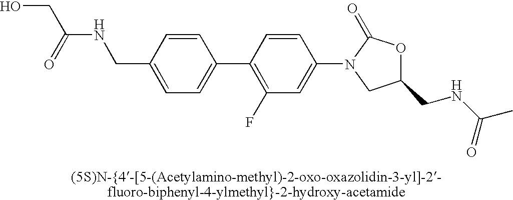 |
| 151 | 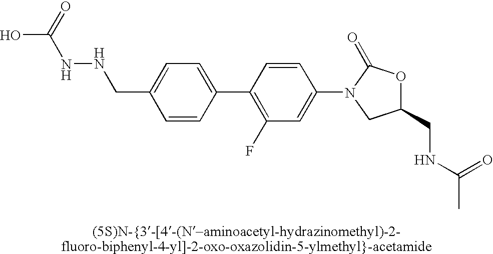 |
| 152 | 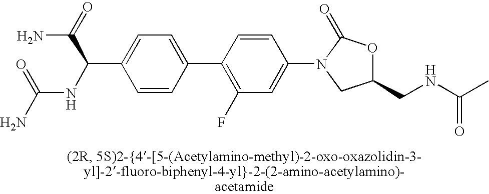 |
| 153 | 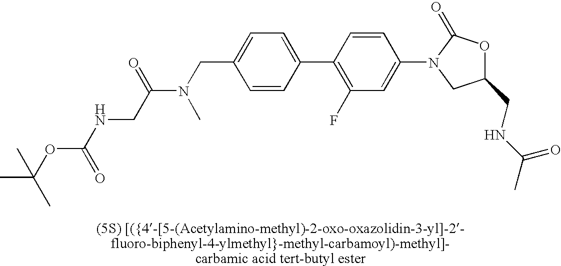 |
| 154 | 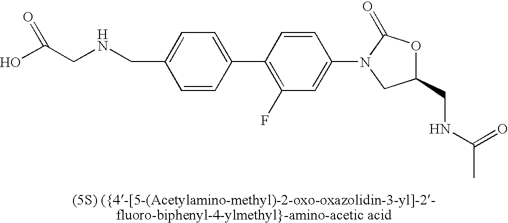 |
| 155 | 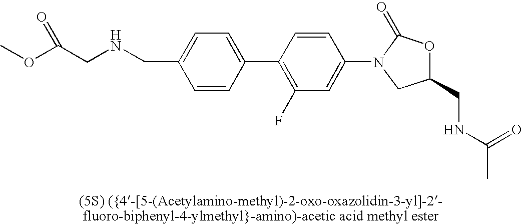 |
| 156 | 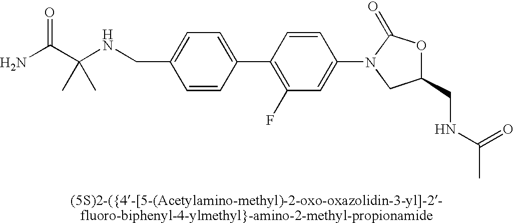 |
| 157 | 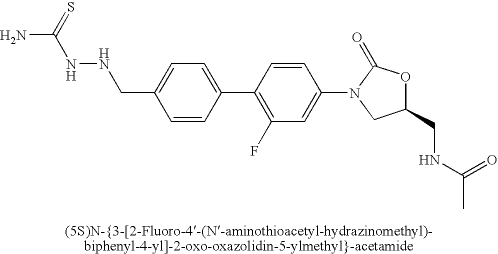 |
| 158 | 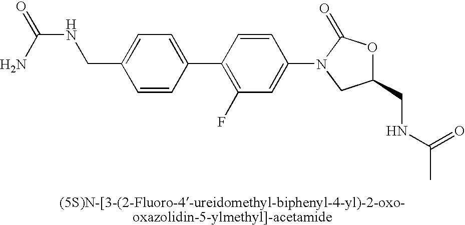 |
| 159 | 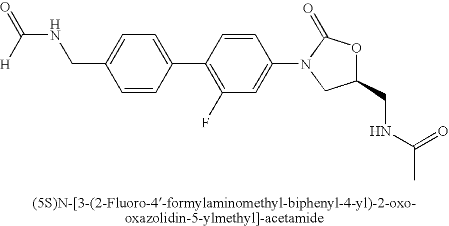 |
| 160 | 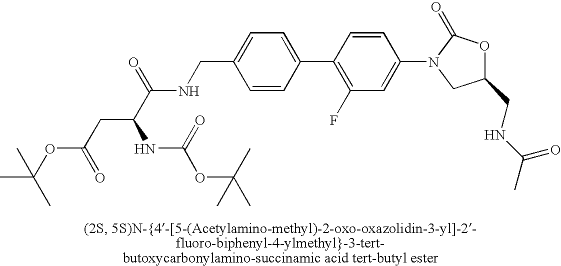 |
| 161 | 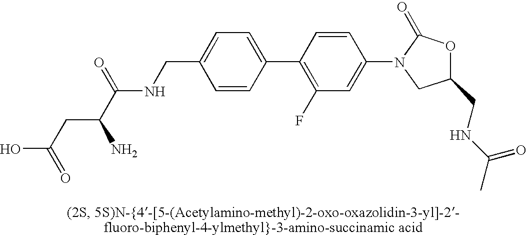 |
| 162 | 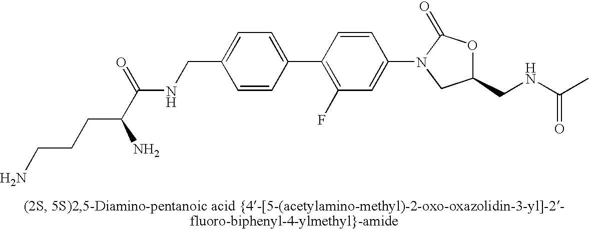 |
| 163 | 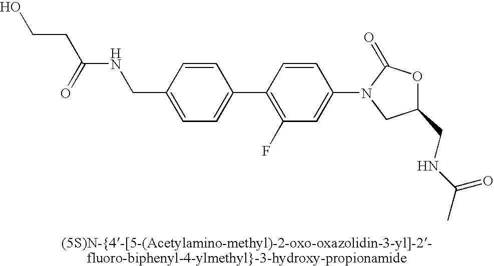 |
| 164 | 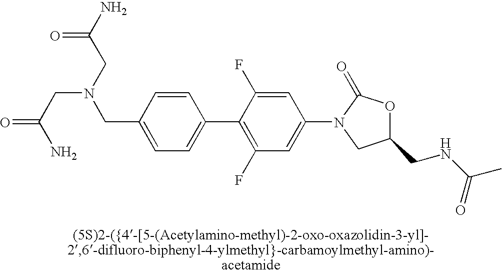 |
| 165 | 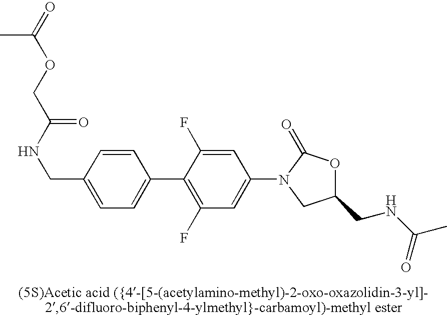 |
| 166 | 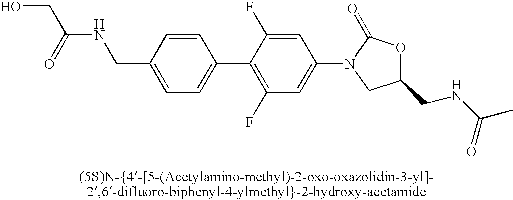 |
| 167 | 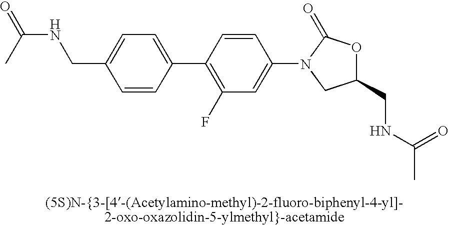 |
| 168 | 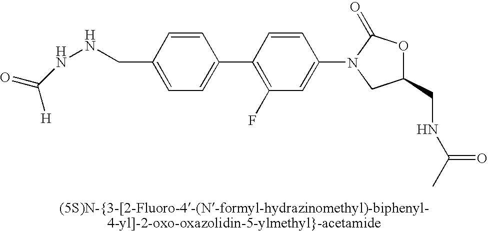 |
| 169 | 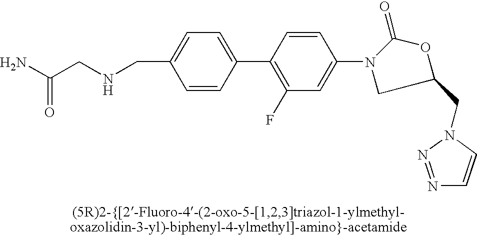 |
| 170 | 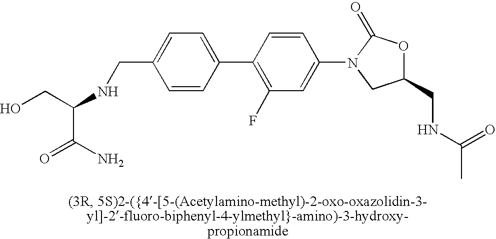 |
| 171 | 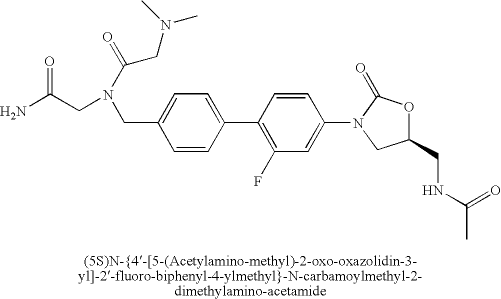 |
| 172 | 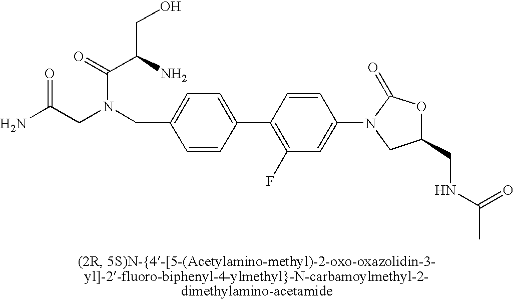 |
| 173 | 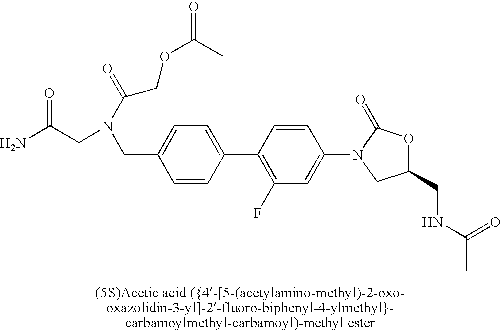 |
| 174 | 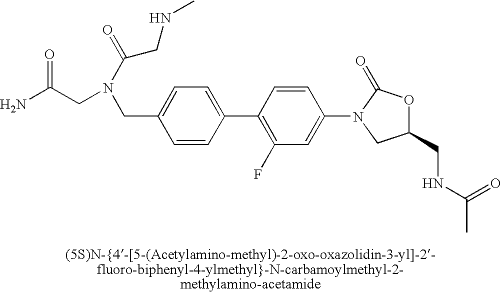 |
| 175 | 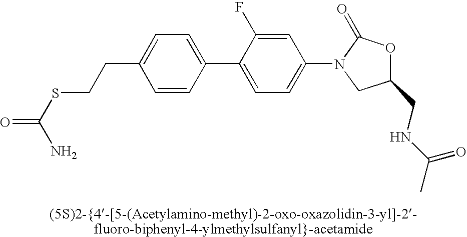 |
| 176 | 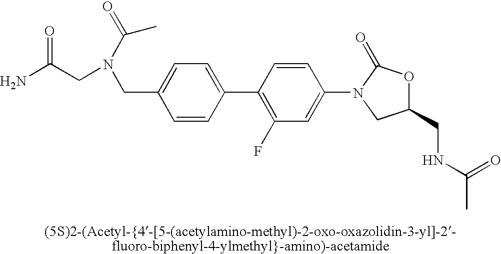 |
| 177 | 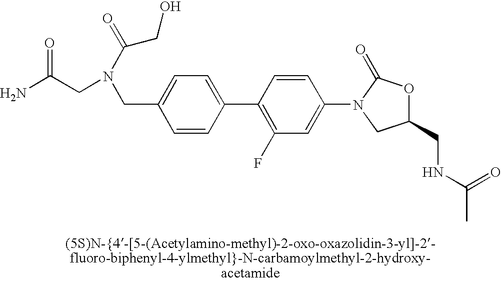 |
| 178 | 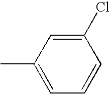 |
| 179 | 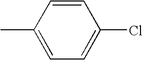 |
| 180 | 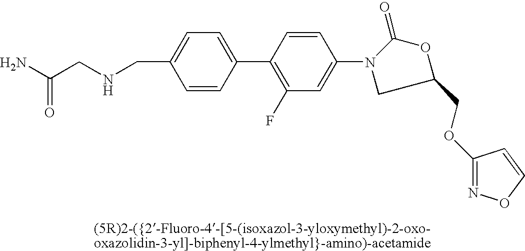 |
| 181 | 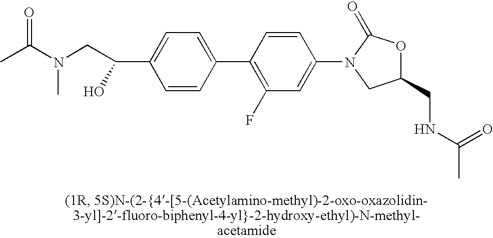 |
| 182 | 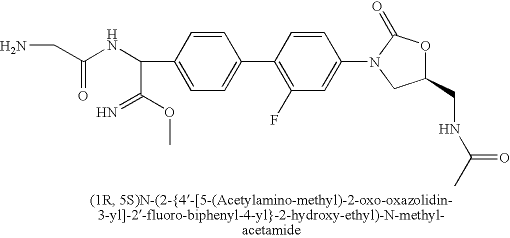 |
| 183 | 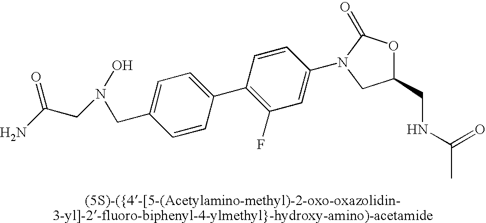 |
| 184 | 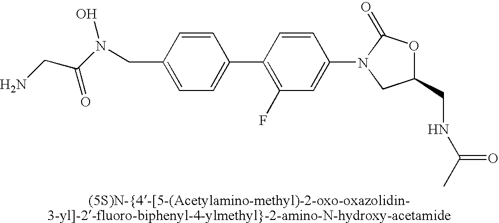 |

The foregoing compounds and others can be straightforwardly synthesized using conventional chemistries known and used in the art. Examples of particular exemplary synthetic schemes are illustrated below in the following non-limiting examples.

### Example 1—Synthesis of Biaryl Precursors

Scheme 1 depicts the synthesis of various biaryl intermediates useful in producing compounds of the present invention. Known iodoaryl oxazolidinone intermediate 1 (see U.S. Pat. Nos. 5,523,403 and 5,565,571) is coupled to a substituted aryl boronic acid (the Suzuki reaction) to produce biaryl alcohol 2. Mesylate 3, azide 4, and amine 5 are then synthesized using chemistry well known to those skilled in the art. 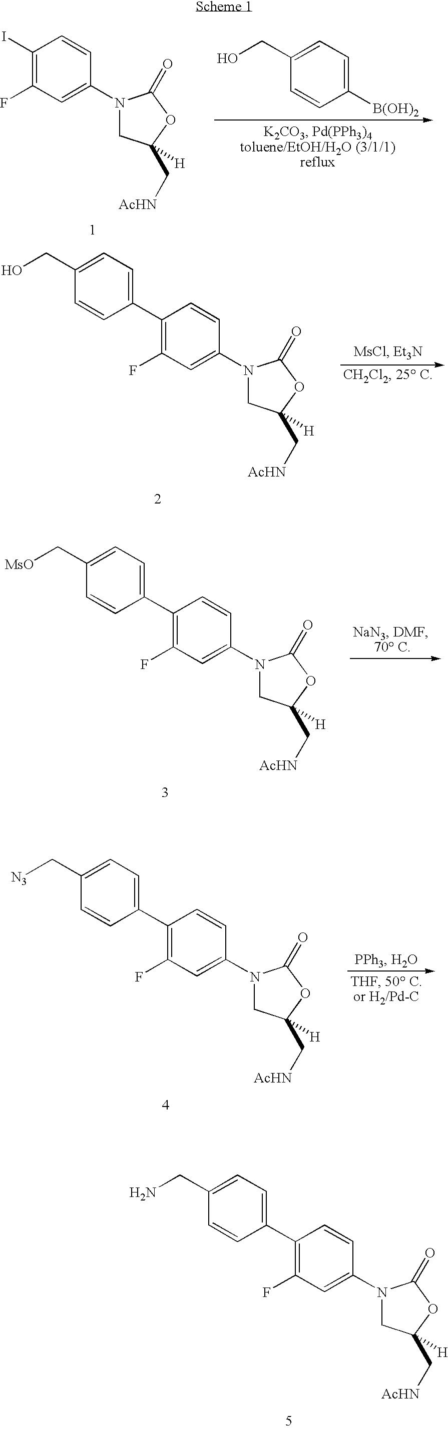
Synthesis of Alcohol 2

A suspension of N-[3-(3-fluoro-4-iodo-phenyl)-2-oxo-oxazolidin-5-ylmethyl]-acetamide 1 (14.0 g, 37 mmol) in toluene (120 mL) was treated with 4-(hydroxymethyl)phenylboronic acid (7.87 g, 51.8 mmol, 1.4 equiv), potassium carbonate (K₂CO₃, 15.32 g, U 1 mmol, 3.0 equiv), ethanol (EtOH, 40 mL), and H₂O (40 mL) at 25° C., and the resulting mixture was degassed three times under a steady stream of argon at 25° C. Tetrakis(triphenylphosphine)palladium (Pd(PPh₃)₄, 2.14 g, 1.85 mmol, 0.05 equiv) was subsequently added to the reaction mixture, and the resulting reaction mixture was degassed three times again before being warmed to gentle reflux for 6 h. When thin layer chromatography (TLC) and HPLC showed the coupling reaction was complete, the reaction mixture was cooled to room temperature before being treated with H₂O (240 mL). The resulting mixture was then stirred at room temperature for 10 min before being cooled to 0-5° C. for 1 h. The solid precipitates were collected by filtration, washed with H₂O (2×100 mL) and 20% ethyl acetate (EtOAc)/hexane (2×50 mL), and dried in vacuo. The crude desired N-[3-(2-Fluoro-4′-hydroxymethyl-biphenyl-4-yl)-2-oxo-oxazolidin-5-ylmethyl]-acetamide 2 (12.50 g, 94% yield) was obtained as off-white solids. This material was found to be essentially pure by HPLC and ¹H NMR and was directly used in the subsequent reaction without further purification. ¹H NMR (300 MHz, DMSO-d₆) δ 1.76 (s, 3H, COCH₃), 3.35 (t, 2H, J=5.4 Hz), 3.69 (dd, 1H, J=6.4, 9.2 Hz), 4.08 (t, 1H, J=9.1 Hz), 4.46 (d, 2H, J=5.7 Hz, CH₂OH), 4.68 (m, 1H), 5.16 (t, 1H, J=5.7 Hz, OR), 7.25-7.52 (m, 7H, aromatic-B), 8.18 (t, 1H, J=5.8 Hz, NHCOCH₃). LCMS (ESI) m/e 359 (M+H)⁺.

Synthesis of Mesylate 3

A suspension of 2 (12.49 g, 34.90 mmol) in methylene chloride (CH₂Cl₂, 150 mL) was treated with triethylamine (Et3N, 7.07 g, 9.7 mL, 70 mmol, 2.0 equiv) at 25° C., and the resulting mixture was cooled to 0-5° C. before being treated dropwise with methanesulfonyl chloride (4.80 g, 3.24 mL, 41.9 mmol, 1.2 equiv) at 0-5° C. The resulting reaction mixture was subsequently stirred at 05° C. for 2 h. When TLC and HPLC showed the reaction was complete, the reaction mixture was treated with H₂O (100 mL) at 0-5° C. The mixture was then concentrated in vacuo to remove most of the CH₂Cl₂, and the resulting slurry was treated with H₂O (150 mL). The mixture was stirred at room temperature for 10 min before being cooled to 0-5° C. for 30 min. The solid precipitates were collected by filtration, washed with H₂O (2×100 mL) and 20% EtOAc/hexane (2×50 mL), and dried in vacuo. The crude desired methanesulfonic acid 4′-[5-(acetylamino-methyl)-2-oxo-oxazolidin-3-yl]-2′-fluoro-biphenyl-4-ylmethyl ester 3 (11.84 g, 78% yield) was obtained as off-white solids, which by TLC and HPLC was found to be essentially pure and was directly used in the subsequent reaction without further purification. LCMS (ESI) m/e 437 (M+H)⁺.

Synthesis of Azide 4

A solution of 3 (9.27 g, 21.26 mmol) in anhydrous N)N-dimethylformamide (DMF, 50 mL) was treated with sodium azide (NaN₃, 5.53 g, 85.04 mmol, 4.0 equiv) at 25° C., and the resulting reaction mixture was warmed to 70-80° C. for 4 h. When TLC and HPLC showed the reaction was complete, the reaction mixture was cooled to room temperature before being treated with H₂O (150 mL). The resulting mixture was stirred at room temperature for 10 min before being cooled to 0-5° C. for 1 h. The solid precipitates were collected by filtration, washed with H₂O (2×100 mL) and 20% EtOAc/hexane (2×50 mL), and dried in vacuo. The crude desired N-[3-(4′-azidomethyl-2-fluoro-biphenyl-4-yl)-2-oxo-oxazolidin-5-ylmethyl]-acetamide 4 (7.16 g, 88% yield) was obtained as off-white solids. The material was found to be essentially pure by TLC and HPLC and was directly used in the subsequent reaction without further purification. LCMS (ESI) m/e 384 (M+H)⁺.

Synthesis of Amine 5

A solution of 4 (7.16 g, 18.69 mmol) in tetrahydrofuran (THF) (100 mL) was treated with triphenylphosphine (PPh₃, 5.88 g, 22.43 mmol, 1.2 equiv) and H₂O (3.6 g, 3.6 mL, 0.2 mmol, 11.0 equiv) at 25° C., and the resulting reaction mixture was warmed to 50-55° C. for 12 h. When TLC and HPLC showed the reduction reaction was complete, the reaction mixture was cooled to room temperature before the solvents were removed in vacuo. The residue was directly purified by flash column chromatography (0-15% methanol (MeOH)—CH₂Cl₂ gradient elution) to afford the desired N-[3-(4′-Aminomethyl-2-fluoro-biphenyl-4-yl)-2-oxo-oxazolidin-5-ylmethyl]-acetamide 5 (5.82 g, 87% yield) as off-white crystals, which were of sufficient purity to be directly used in subsequent reactions. ¹H NMR (300 MHz, DMSO-d₆) δ 1.85 (s, 3H, COCH₃), 3.04 (br. s, 2H, NH₂), 3.44 (t, 2H, J=5.4 Hz), 3.78 (m, 3H), 4.18 (t, 1H, J=9.1 Hz), 4.77 (m, 1H), 7.25-7.60 (m, 7H, aromatic-B), 8.20 (t, 1H, J=5.8 Hz, NHCOCH₃). LCMS (ESI) m/e 359 (M+2H)²⁺.

### Example 2—Synthesis of Compound 101

Compound 101 was synthesized from amine 5 and bromoacetamide 6 as shown in Scheme 2 below.

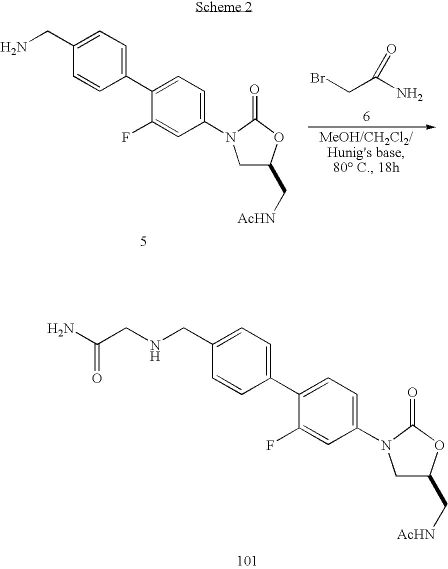

Method A: A mixture of amine 5 (0.075 g, 0.21 mmol) and bromoacetamide 6 (0.030 g, 0.21 mmol) in anhydrous CH₂Cl₂ (2 mL), MeOH (2 ML) and N,N-diisopropylethylamine (Hunig's base, 2 mL) was heated at 80° C. for 18 h. Solvent was removed in vacuo and the crude product was purified on a silica gel column (CH₂Cl₂/MeOH/NH₄OH, 20:1:0.05 to 18:1:0.05 to 16:1:0.05 to 14:1:0.05) to yield compound 101 as white solid (0.064 g, 74%).

An alternative synthesis of compound 101 from aldehyde 7 and glycinamide hydrochloride 8 is shown in Scheme 3 below.

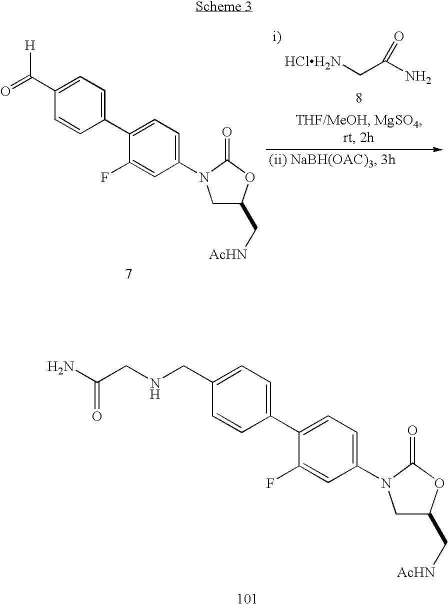

Method B: To a suspension of glycinamide hydrochloride 8 (0.076 g, 0.674 mmol) and magnesium sulfate (MgSO₄, 0.250 g, 2.080 mmol) in MeOH (4 mL) and THF (1 mL) was added oxazolidinone aldehyde 7 (made from iodide 1 and 4-formylboronic acid in the same fashion as the synthesis of alcohol 2 in Example 1) (0.120 g, 0.337 mmol). The mixture was stirred 2 h at room temperature. Sodium triacetoxyborohydride (NaBH(OAc)₃, 0.200 g, 0.940 mmol) was added and stirring continued for 3 h. The reaction was filtered and solvent was removed in vacuo. The crude was purified as in Method A above to give 101 as white solid (0.026 g, 19%). ¹H-NMR, (300 MHz, DMSO-d₆) δ 8.27 (t, J=6 Hz, 1H), 7.65-7.39 (m, 7H), 7.32 (bs, 1H), 7.07 (bs, 1H), 4.76 (m, 1H), 4.17 (t, J=9 Hz, 1H), 3.81-3.72 (m, 3H), 3.43 (t, J=5 Hz, 2H), 3.05 (s, 2H), 1.84 (s, 3H). LCMS (ESI) m/e 415.2 (M+H)⁺.

### Example 3—Synthesis of Compound 102

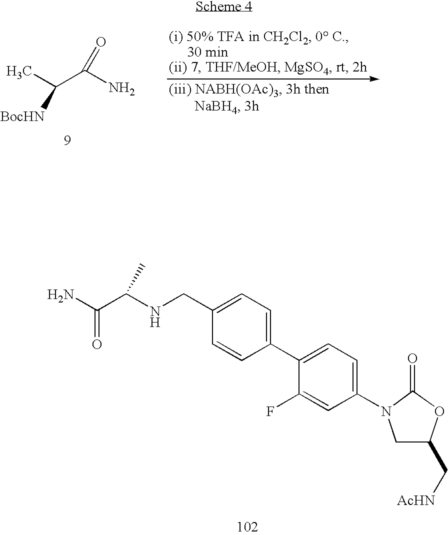

Compound 102 was synthesized by first treating Boc-alanamide 9 with 50% trifluoroacetic acid (TFA) in CH₂Cl₂ at 0° C. for 30 min. After the solvent was removed in vacuo, the crude Boc-deprotected product was reacted with aldehyde 7 as described in Method B for the synthesis of 101, except that NaBH₄ was used as the hydride source. Compound 102 was isolated as white solid in about 1.4% yield after column purification (CH₂Cl₂/MeOH/NH₄OH, 25:1:0.05 to 20:1:0.05 to 15:1:0.05). ¹H-NMR, (300 MHz, CDCl₃/CD₃OD) δ 7.53-7.25 (m, 7H), 4.73 (m, 1H), 4.10 (t, J=9 Hz, 1H), 3.79-3.57 (m, 3H), 3.49 (d, J=5 Hz, 2H), 1.88 (s, 3H), 1.20 (d, J=7 Hz, 3H). LCMS (ESI) m/e 429.2 (M+H)⁺.

### Example 4—Synthesis of Compound 103

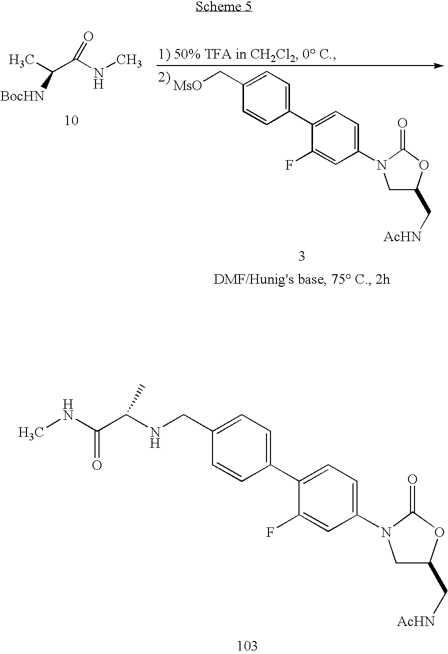

Amide 10 (0.101 g, 0.5 mmol) was dissolved in CH₂Cl₂ (2 mL) at 0° C. Trifluoroacetic acid (2 mL) was added and the mixture was stirred for 30 min at 0° C., at which point the solvent was removed in vacuo. To the crude was added mesylate 3 (0.100 g, 0.23 mmol), DMF (3 mL) and Hunig's base (0.3 mL), and the mixture was heated between 75-80° C. for 2 h. The solvent was removed in vacuo and the crude was partitioned between saturated NaHCO₃ (30 mL) and 15% MeOH in CH₂Cl (30 mL). The aqueous layer was back extracted with 15% MeOH in CH₂Cl₂ (2×30 mL), the combined organic layer was dried over sodium sulfate (Na₂SO₄), and the solvent removed in vacuo. The crude was purified on silica gel (CH₂Cl₂/MeOH/NH₄OH, 20:1:0.05 to 15:1:0.05) to give compound 103 as white hygroscopic solid (0.032 g, 32%). 1H-NMR, (300 MHz, CDCl₃/CD₃OD) δ 7.70-7.28 (m, 7H), 4.82 (m, 1H), 4.15 (t, J=9 Hz, 1H), 3.87-3.59 (m, 5H), 3.22 (m, 1H), 2.78 (s, 3H), 2.00 (s, 3H), 1.29 (d, J=7 Hz, 3H). LCMS (ESI) m/e 443.2 (M+H)⁺.

### Example 5—Synthesis of Compound 104

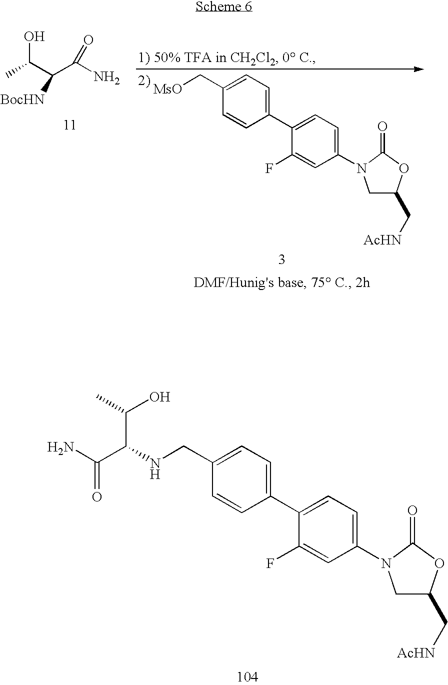

Compound 104 was synthesized from amide 11 and mesylate 3 as described for compound 103, except that the crude was directly purified on silica gel after solvent evaporation, eluting with CH₂Cl₂/MeOH/NH₄OH (20:1:0.05 to 15:1:0.05 to 12:1:0.05), to give compound 104 as a white solid. ¹H-NMR, (300 MHz, CDCl₃/CD₃OD) δ 7.51-7.21 (m, 7H), 4.72 (m, 1H), 4.08 (t, J=9 Hz, 1H), 3.83-3.41 (m, 6H), 2.93 (m, 1H), 1.92 (s, 3H), 1.75 (d, J=7 Hz, 3H). LCMS (ESI) m/e 459.3 (M+H)⁺.

### Example 6—Synthesis of Compound 105

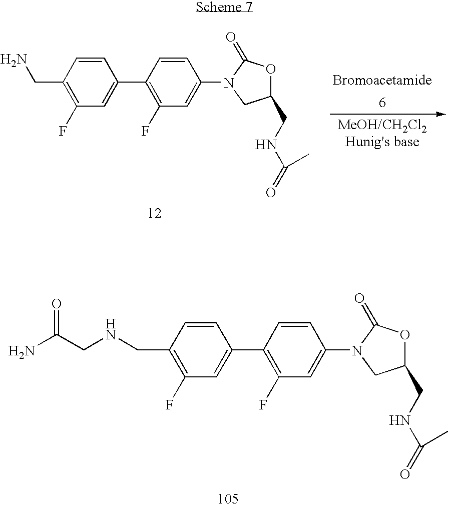

To a solution of amine 12 (made from iodide 1 and 4-formyl-3-fluorophenylboronic acid in the same fashion as the synthesis of amine 5 in Example 1) (86 mg, 0.23 mmol) in a mixture of methyl alcohol (2 mL), methylene chloride (2 mL) and Hunig's base (2 mL) was added bromoacetamide 6 (32 mg, 0.23 mmol) at 0° C. The reaction mixture was warmed to room temperature and heated over an oil bath at 80° C. for 18 h. The solution was concentrated and purified by flash chromatography (14:1:0.05 CH₂Cl₂/MeOH/NH₄OH) to yield 66 mg of compound 105. HNM (300 MHz, DMSO): δ 8.27 (t, J=6 Hz, 1H), 7.65-7.54 (m, 3H), 7.44-7.35 (m, 4H), 7.14 (s, 1H), 4.81-4.72 (m, 1H), 4.17 (t, J=9 Hz, 1H), 3.80-3.71 (m, 3H), 3.43 (t, J=5 Hz, 2H), 3.13 (s, 2H), 1.84 (s, 3H). LCMS (ESI) mne 433 (M+H)⁺.

### Example 7—Synthesis of Compound 106

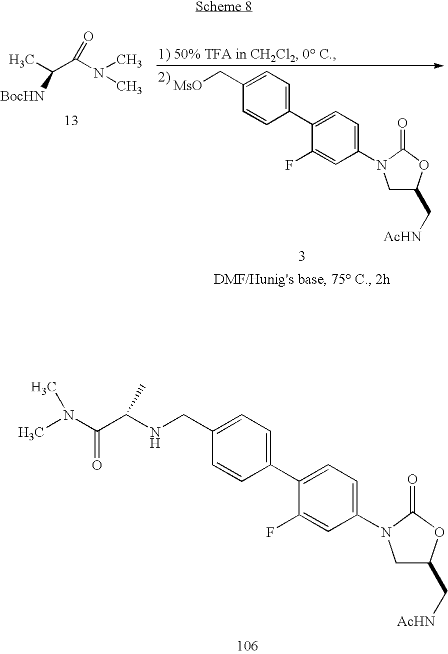

Compound 106 was synthesized from amide 13 and mesylate 3 as described for compound 103. The crude was purified on silica gel (first eluting with CH₂Cl₂/MeOH, 24:1 to 20:1, then with CH₂Cl₂/MeOH/NH₄OH, 20:1:0.05 to 15:1:0.05) to give compound 106 as a white hygroscopic solid. ¹H-NMR, (300 MHz, CDCl₃) δ 7.47-7.21 (m, 7H), 5.98 (t, J=6 Hz, 1H), 4.74 (m, 1H), 4.02 (t, J=9 Hz, 1H), 3.78-3.48 (m, 6H), 2.93 (s, 3H), 2.91 (s, 3H), 1.97 (s, 3H), 1.17 (d, J=7 Hz, 3H). LCMS (ESI) m/e 457.3 (M+H)⁺.

### Example 8—Synthesis of Compound 107

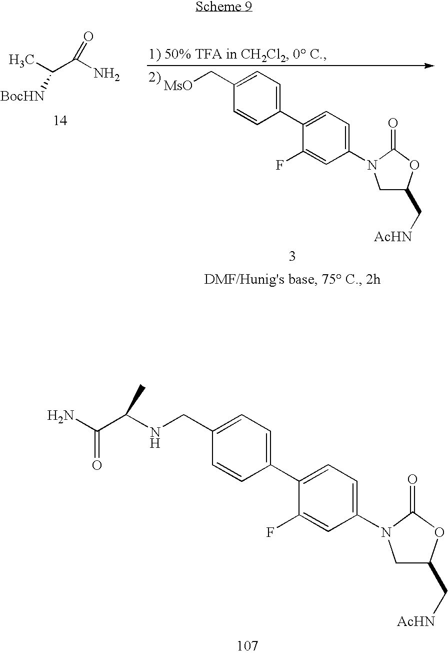

Compound 107 was synthesized from amide 14 and mesylate 3 as described for compound 103, except that the reaction was heated for 12 h. The crude was purified on silica gel (CH₂Cl₂/MeOE/NH₄OH, 30:1:0.04 to 25:1:0.04 to 20:1:0.04 to 15:1:0.05 to 15:1:0.1) to give compound 107 as white hygroscopic solid. ¹H-NMR, (300 MHz, CDCl₃/CD₃OD) δ 7.48-7.20 (m, 7H), 4.74 (m, 1H), 4.07 (t, J=9 Hz, 1H), 3.79-3.50 (m, 5H), 3.19 (m, 1H), 1.92 (s, 3H), 1.25 (d, J=7 Hz, 3H). LCMS (ESI) m/e 429.2 (M+H)⁺.

### Example 9—Synthesis of Compound 108

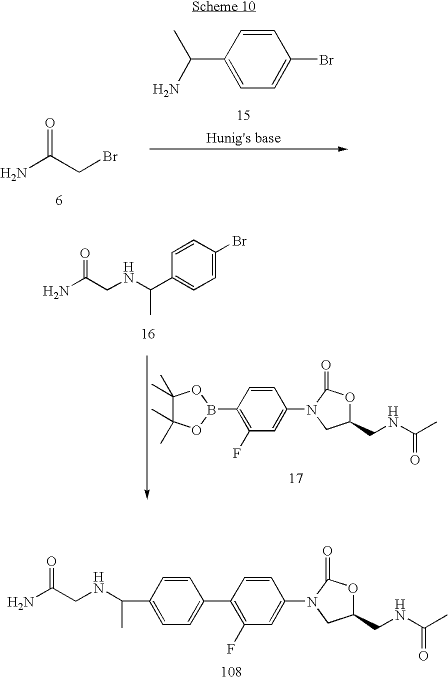

To a solution of 2-bromoacetamide 6 (827 mg, 5.88 mmol), and 4-bromobenzyl-ethylamine 15 (1.00 g, 4.90 mmol) in MeOH (5 mL) and CH₂Cl₂ (5 mL) at room temperature was added Hunig's base (5 mL). The mixture was stirred at 50-60° C. for 16 h. Water (30 mL) was added and the mixture was extracted with CH₂Cl₂ (4×30 mL) and dried over Na₂SO₄ to provide 1.27 g amide 16 as white crystals in 100% yield. This product was used without further purification in the next step. ¹H NMR (300 MHz, CDCl₃, ppm): δ: 7.38 (d, J=8 Hz, 2H), 7.09 (d, J=8 Hz, 1H), 6.77 (s br, 1H), 5.69 (s br, 1H), 3.67 (q, J=7 Hz, 1H), 3.07 (s, 2H), 1.29 (d, J=7 Hz, 3H).

Boronic ester 17 (synthesized as described in PCT patent publication WO 03/027083A1) (168 mg, 0.4 mmol) was added to a mixture of amide 16 (103 mg, 0.40 mmol), dichloro[1,1′-bis(diphenylphosphino)ferrocene]palladium(I) (Pd(dppf)₂Cl₂, 16 mg, 0.02 mmol) and K₂CO₃ (221 mg, 1.60 mmol) in dioxane (3 mL), EtOH (1 mL) and H₂O (1 mL). The mixture was degassed with argon and stirred at 90-95° C. for 3 h. Water (10 mL) was added, the mixture was extracted with CH₂Cl₂ (4×30 mL), and the extracts were dried over Na₂SO₄. The residue was purified by column chromatography (7:100:0.1 MeOH/CH₂Cl₂/NH₄OH) to give 85 mg compound 108 in 50% yield. ¹HNMR (300 MHz, CDCl₃, ppm, partial): δ: 7.70-7.30 (m, 7H), 7.09 (s br, 1H), 6.31 (s br, 1H), 5.63 (s br, 1H), 4.96-4.92 (m, 1H), 4.22 (t, J=9 Hz, 1H), 3.33 (s, 2H), 2.13 (s, 3H), δ 1.55 (d, J=7 Hz, 3H). LCMS (ESI) m/e 451.2 (M+Na)⁺.

### Example 10—Synthesis of Compound 109

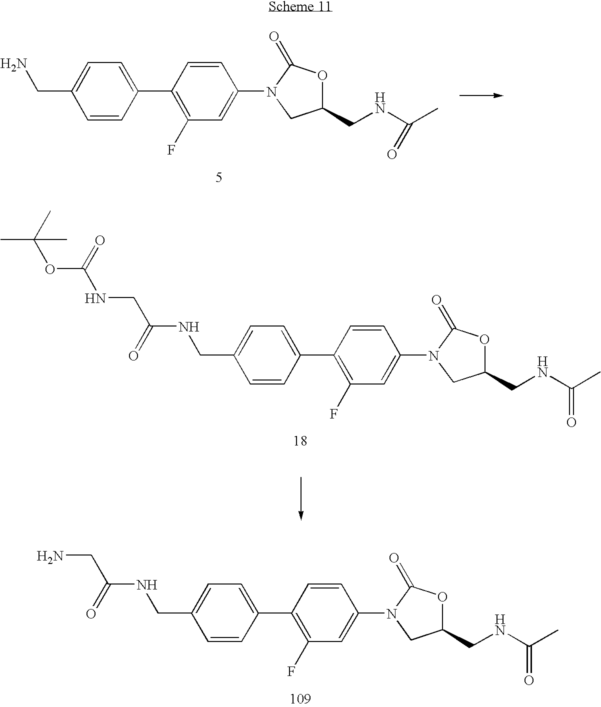

A suspension of polymeric 4-hydroxy-2,3,5,6-tetrafluorophenol amide resin (TFP, J. Comb. Chem. 2000, 2, 691) (1.00 g, 1.27 mmol) in DMF (10 mL) was shaken for 10 minutes in a 70 mL polypropylene cartridge and then treated with N-tert-Butoxycarbonyl-glycine (1.11 g, 6.35 mmol), 3-hydroxybenzotriazole (18 mg, 0.13 mmol), and diisopropylcarbodiimide (1.2 mL, 7.6 mmol). The reaction mixture was shaken for 18 h at 23° C., and then the resin was washed with DMF (10×50 mL), THF (10×50 mL), and methylene chloride (10×50 mL) and dried in vacuo.

A suspension of TFP ester (270 mg, 0.216 mmol) in 7 mL DMF was treated with amine 5 (70 mg, 0.196 mmol) and shaken for 18 h in a 10 mL polypropylene cartridge. The filtrate was collected and dried to give amide 18 (90 mg, 0.175 mmol, 89%). ¹HNMR (300 MHz, DMSO-d₆): δ 8.44-8.40 (m, 1H), 8.37-8.33 (m, 1H), 7.68-7.53 (m, 4H), 7.49-7.40 (m, 3H), 7.12-7.08 (m, 1H), 4.87-4.79 (m, 1H), 4.39 (d, J=6 Hz, 2H), 4.26-4.20 (m, 1H), 3.84 (dd, J=9, 6 Hz, 1H), 3.66-3.62 (m, 2H), 3.52-3.48 (m, 2H), 1.91 (s, 3H), 1.46 (s, 9H).

A solution of amide 18 (90 mg, 0.175 mmol) in 4 M HCl in dioxane (1 mL) was stirred at 23° C. for 0.5 h. The solvent was removed in vacuo. The reaction was twice diluted with dichloromethane (5 mL) and the solvent evaporated in vacuo to provide the hydrochloride salt of compound 109 (57 mg, 0.138 mmol, 79%). MS (ESI): 415.1 (M+H)⁺. ¹HNMR (300 MHz, DMSO-d₆): δ 9.06-9.02 (m, 1H), 8.40-8.36 (m, 1H), 8.20 (s, 3H), 7.68-7.56 (m, 4H), 7.49-7.45 (m, 3H), 4.87-4.78 (m, 1H), 4.46-4.44 (m, 2H), 4.26-4.20 (m, 1H), 3.85 (dd, J=9, 6 Hz, 1H), 3.72-3.65 (m, 2H), 3.51-3.47 (m, 2H), 1.90 (s, 3H).

### Example 11—Synthesis of Compound 110

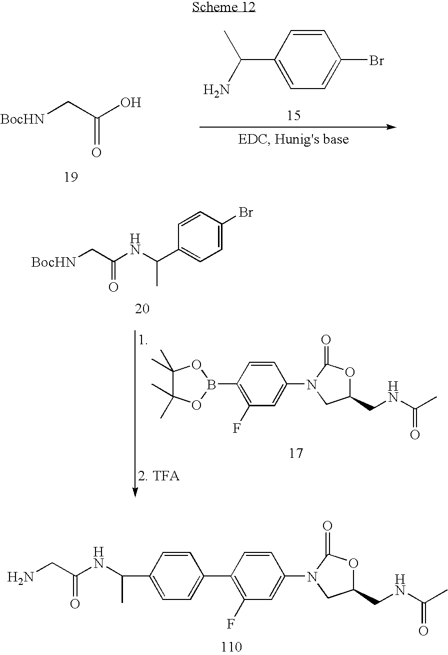

To a solution of Boc-glycine 19 (1.04 g, 5.88 mmol) and 4-bromobenzylethylamine 15 (1.00 g, 4.90 mmol) in CH₂Cl₂ (25 mL) at room temperature was added Hunig's base (1.30 mL, 7.35 mmol). The mixture was stirred at room temperature for 16 h. The mixture was poured into water (40 mL) and saturated NaHCO₃ (3 mL), then extracted with CH₂Cl₂ (60 mL), washed with water (100 mL), and dried over Na₂SO₄. The residue was purified by column chromatography (50:50:0.1 EtOAc/Hexane/NH₄OH), to give 1.30 g amide 20 as white crystals in 69% yield. ¹HNMR (300 MHz, CDCl₃, ppm): δ: 7.37 (d, J=7 Hz, 2H), 7.09 (d, J=7 Hz, 1H), 6.50 (s br, 1H), 5.15 (s br, 1H), 5.01-4.95 (m, 1H), 3.69 (d, J=6 Hz, 2H), 1.39 (d, J=7 Hz, 3H), 1.37 (s, 9H).

A mixture of 20 (143 mg, 0.40 mmol), boronic ester 17 (168 mg, 0.4 mmol), Pd(dppf)₂Cl₂ (16 mg, 0.02 mmol) and K₂CO₃ (221 mg, 1.60 mmol) in dioxane (3 mL), EtOH (1 mL) and H₂O (1 mL) was degassed with argon. The mixture was stirred at 90-95° C. for 3 h. Water (10 mL) was added and the mixture was extracted with CH₂Cl₂ (4×30 mL), and the organic extracts dried over Na₂SO₄. The residue was purified by column chromatography (5:100:0.1 MeOH/CH₂Cl₂/NH₄OH) to give 200 mg of Boc-protected product in 100% yield. To this intermediate was added CH₂Cl₂ (2 mL) and TFA (2 mL), and the mixture was stirred at room temperature for 3 hr. The solvent was removed in vacuo and the residue was purified by column chromatography (15:85:0.1 MeOH/CH₂Cl₂/NH₄OH) to give 168 mg of compound 110 in 99% yield. ¹HNMR (300 MHz, CDCl₃, ppm, partial): δ: 7.64-7.33 (m, 7H), 5.13-5.07 (m, 1H), 4.19 (ddd, J=9, 3, 3 Hz, 1H), 3.87 (ddd, J=9, 7, 3 Hz, 1H), 3.77-3.50 (m, 5H), 1.99 (s, 3H), 1.54 (d, J=7 Hz, 3H). LCMS (ESI) m/e 429 (M+H)⁺.

### Example 12—Synthesis of Compound 111

A solution of amine 5 (70 mg, 0.20 mmol) in DMF (1 mL) was treated with diisopropylethylamine (0.068 mL, 0.39 mmol), and then cooled to 0° C. The reaction mixture was treated with N,N-dimethylglycine (20 mg, 0.20 mmol) and 1-(3-dimethylaminopropyl)-3-ethylcarbodiimide hydrochloride (EDCI, 41 mg, 0.22 mmol). The reaction was stirred at 0° C. for 2 h. Additional N,N-dimethylglycine (20 mg, 0.20 mmol) and EDCI (34 mg, 0.20 equiv.) were added, and the reaction was stirred at 23° C. for 24 h. 3-hydroxybenzotriazole (29 mg, 0.22 mmol) was added, and the reaction was stirred at 23° C. for 16 h. The reaction was diluted with toluene and the solvent removed in vacuo. The crude product was purified via preparative thin-layer chromatography (10:1:0.1 CH₂Cl₂/MeOH/NH₄OH) to provide 7 mg compound 111 in 8% yield. MS (ES): 443.1 (M+H)⁺. ¹HNMR (300 MHz, CH₂Cl₂): δ 7.56-4.47 (m, 4H), 7.43-7.33 (m, 3H), 7.24-7.23 (m, 1H), 6.35-6.45 (m, 1H), 4.84-4.76 (m, 1H), 4.52-4.50 (m, 2H), 4.10-4.04 (m, 1H), 3.81 (dd, J=9, 7 Hz, 1H), 3.71-3.61 (m, 2H), 3.02 (s, 2H), 2.29 (s, 6H), 2.02 (s, 3H).

### Example 13—Synthesis of Compound 113

A suspension of polymeric 4-hydroxy-2,3,5,6-tetrafluorophenol (TFP, J. Comb. Chem. 2000, 2, 691) amide resin (1.00 g, 1.27 mmol) in DMF (10 mL) was shaken for 10 minutes in a 70 mL polypropylene cartridge and then treated with N-tert-Butoxycarbonyl-glycine (1.11 g, 6.35 mmol), 3-hydroxybenzotriazole (18 mg, 0.13 mmol), and diisopropylcarbodiimide (1.2 mL, 7.6 mmol). The reaction mixture was shaken for 18 h at 23° C., and then the resin was washed with DMF (10×50 mL), THF (10×50 mL), and methylene chloride (10×50 mL) and dried in vacuo.

A suspension of the N-Boc glycine-loaded TFP ester resin (0.051 g, 0.041 mmol) in DMF (1 mL) was treated with amine 12 (0.014 g, 0.037 mmol) and shaken for 12 h in a 7 mL polypropylene cartridge. The filtrate was collected and evaporated to give 17 mg crude Boc-protected product, which was taken up in dichloromethane (2 mL) and methanol (2 mL) and treated with 4.0 M HCl in dioxane (5 mL). The reaction was stirred at 23° C. for 2 h. Removal of the solvent in vacuo yielded compound 113 as the hydrochloride salt (0.015 g, 0.032 mmol, 87% for 2 steps): MS (ESI): 433 (M+H)⁺.

### Example 14—Synthesis of Compound 114

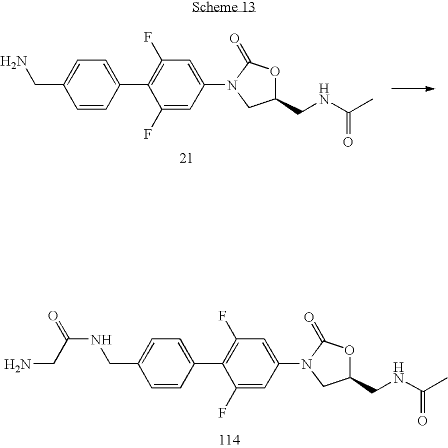

A solution of amine 21 (0.079 g, 0.21 mmol) in DMF (2 mL) was treated with N-Boc-glycine (0.054 g, 0.31 mmol) and EDCI (0.060 g, 0.31 mmol) and stirred at 23° C. for 2 h. The reaction mixture was diluted with dichloromethane (50 mL), washed with 1 M hydrochloric acid (2×50 mL) and saturated aqueous NaHCO₃ (50 mL), and dried over Na₂SO₄. The solvent was removed in vacuo to provide Boc-protected product (89 mg) as a white powder. A solution of the crude Boc-protected product in dichloromethane (15 mL) and methanol (5 ni) was treated with 4.0 M HCl in dioxane (5 mL) and stirred at 23° C. for 2 h. Removal of the solvent in vacuo yielded 0.065 g compound 114 as the hydrochloride salt in a 66% yield. MS (ESI): 433 (M+H)⁺.

### Example 15—Synthesis of Compound 117

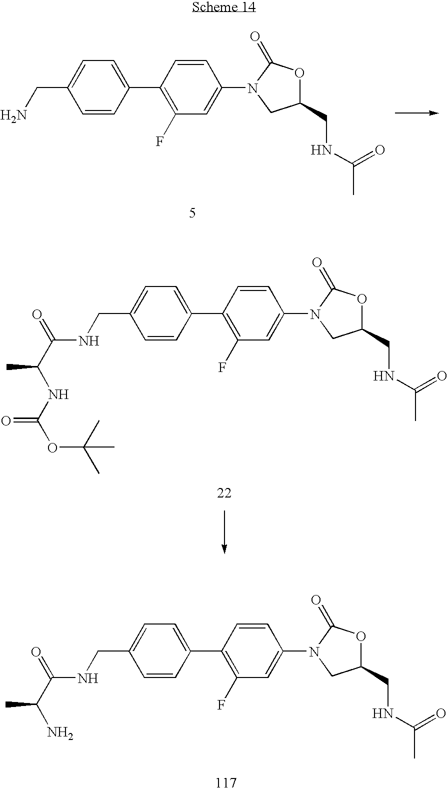

A suspension of amine 5 (0.070 g, 0.20 mmol) in DMF (1.0 mL) was treated with diisopropylethylamine (0.068 mL, 0.40 mmol) and heated until dissolution was achieved. The reaction mixture was cooled to 0° C. and treated with N-tert-butoxycarbonyl-alanine (0.037 g, 0.20 mmol) and EDCI (0.041 g, 0.22 mmol). Additional N-tert-butoxycarbonyl-alanine (0.037 g, 0.20 mmol) and EDCI (0.034 g, 0.18 mmol) were added and the reaction mixture was stirred at 23° C. for 12 h. 1-hydroxybenzotriazole (0.029 g, 0.22 mmol) was added. The reaction mixture was diluted in toluene and the solvent removed in vacuo to yield crude product, which was purified by flash chromatography (2-5% MeOH in 1:1 EtOAc/CH₂Cl₂) to afford 0.053 g amide 22 in 50% yield.

A solution of amide 22 (0.042 g, 0.080 mmol) in methylene chloride (1.0 mL) was treated with hydrogen chloride (4.0 M in dioxane, 0.24 mL) and stirred at 23° C. for 1 h. The reaction mixture was diluted with methylene chloride and the solvent removed in vacuo to yield compound 117. MS (ESI): 429 (M+H)⁺.

### Example 16—Synthesis of Compound 119

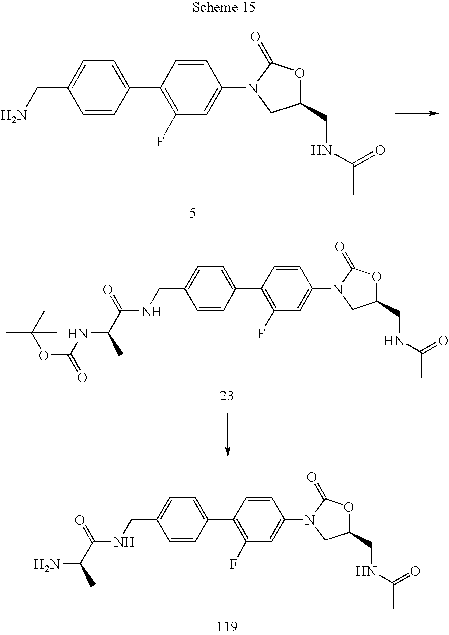

A solution of amine 5 (0.020 g, 0.056 mmol) in DMF (0.56 mL) was treated with N-tert-butoxycarbonyl-D-alanine (0.012 g, 0.062 mmol) and EDCI (0.016 g, 0.084 mmol) and stirred at 23° C. for 1.5 h. The reaction mixture was diluted with methylene chloride (15 mL), washed with 1.0 M hydrochloric acid (2×15 mL) and saturated aqueous sodium bicarbonate (15 mL), and dried with Na₂SO₄ to yield amide 23.

A solution of amide 23 (0.045 g, 0.085 mmol) in methylene chloride (1.0 mL) was treated with hydrogen chloride (4.0 M in dioxane, 0.50 mL) and stirred at 23° C. for 1 h. The reaction mixture was diluted with methylene chloride and the solvent removed in vacuo to yield compound 119. MS (ESI): 429 (M+H)⁺.

### Example 17—Synthesis of Compound 126

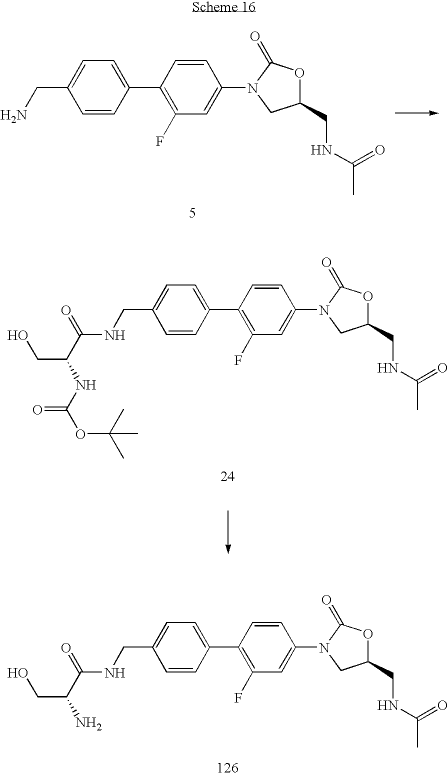

A solution of amine 5 (0.050 g, 0.14 mmol) in DMF (0.50 mL) was treated with N-tert-butoxycarbonyl-D-serine (0.032 g, 0.15 mmol), 4-dimethylaminopyridine (DMAP, 0.8 M in DMF, 0.17 mL, 0.14 mmol), and EDCI (0.030 g, 0.15 mmol) and stirred at 23° C. for 0.5 h. The reaction mixture was added directly to a silica column and purified by flash chromatography (50-100% acetone in hexane) to afford 0.038 g amide 24 in 48% yield.

A solution of amide 24 (0.038 g, 0.067 mmol) in methylene chloride (1.0 mL) was treated with hydrogen chloride (4.0 M in dioxane, 0.80 mL) and stirred at 23° C. for 1 h. The reaction mixture was diluted with methylene chloride and the solvent removed in vacuo to yield compound 126. MS (ESI): 445 (M+H)⁺.

### Example 18—Synthesis of Compound 127

A solution of amine 5 (0.025 g, 0.07 mmol) in DMF (0.5 mL) was treated with N-Boc-glutamic acid (0.054 g, 0.08 mmol), DMAP (0.010 g, 0.08 mmol.) and EDCI (0.016 g, 0.08 mmol) and stirred at 23° C. for 2 h. The reaction mixture was diluted with dichloromethane (10 mL), washed with 1 M HCl (2×10 mL) and saturated aqueous NaHCO₃ (10 mL), and dried over Na₂SO₄. The solvent was removed in vacuo to provide the Boc-protected product (62 mg) as a white solid which was further purified by silica gel chromatography (acetone) to afford the pure Boc-protected amine as a white powder (41 mg). This solid was taken up in 0.5 mL THF, cooled to 0° C. and treated with 0.1 mL 2N HCl in dioxane. The mixture was stirred 4 h at 0° C., then concentrated to give the hydrochloride salt of compound 127 as a hygroscopic white solid (0.029 g, 0.06 mmol, 86% for 2 steps). MS (ESI): 486 (M+H)⁺.

### Example 19—Synthesis of Compound 129

A solution of amine 5 (0.025 g, 0.07 mmol) in DMF (0.5 mL) was treated with N-Boc-L-methionine (0.020 g, 0.08 mmol), DMAP (0.010 g, 0.08 mmol) and EDCI (0.016 g, 0.08 mmol) and stirred at 23° C. for 2 h. The reaction mixture was diluted with dichloromethane (10 mL), washed with 1 M HCl (2×10 mL) and saturated aqueous NaHCO₃ (10 mL), and dried over Na₂SO₄. The solvent was removed in vacuo to provide the Boc-protected product (62 mg) as a white solid which was further purified by silica gel chromatography (acetone) to afford the pure Boc-protected amine as a white powder (34 mg). This solid was taken up in 0.5 mL THF, cooled to 0° C. and treated with 0.1 mL 2N HCl in dioxane. The mixture was stirred 4 h at 0° C. and then concentrated to give the hydrochloride salt of compound 129 as a hygroscopic white solid (0.027 g, 0.055 mmol, 79% for 2 steps). MS (ESI): 489 (M+H)⁺.

### Example 20—Synthesis of Compound 138

To a 0° C. solution of compound 129 (0.010 g, 0.02 mmol) in dichloromethane (0.5 mL) and methanol (0.5 mL) was added 3-chloroperoxybenzoic acid (0.005 g, 0.03 mmol), and the solution was stirred at 0° C. for 6 h. The solution was diluted with dichloromethane (30 mL) and washed with saturated aqueous NaHCO₃ and brine. The crude product was purified by silica gel chromatography (acetone) to yield compound 138 as a white solid (0.011 g, 0.02 mmol, 100% yield). MS (ESI): 521 (M+H)⁺.

### Example 21—Synthesis of Compound 146

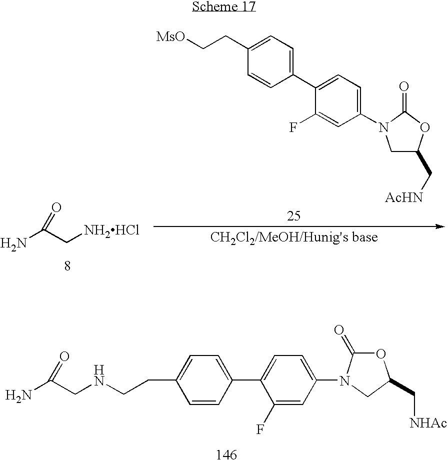

A mixture of glycinamide hydrochloride 8 (0.40 g, 3.40 mmol) and mesyl-oxazolidinone 25 (0.50 g, 1.10 mmol) in anhydrous CH₂Cl₂ (10 mL), MeOH (10 mL) and Hunig's base (4 mL) was heated between 80° C. to 85° C. for 60 h. The solvent was removed in vacuo and the crude was suspended in dilute ammonium hydroxide (pH=10) and filtered. The residue was dried and purified by silica gel chromatography (1% MeOH in CH₂Cl₂, then 16:1:0.1 to 12:1:0.1 CH₂Cl₂/MeOH/NH₄OH) to give compound 146 as white solid (0.192 g, 41%). LCMS (ESI): m/e 429.1 (+H)⁺.

### Example 22—Synthesis of Compound 148

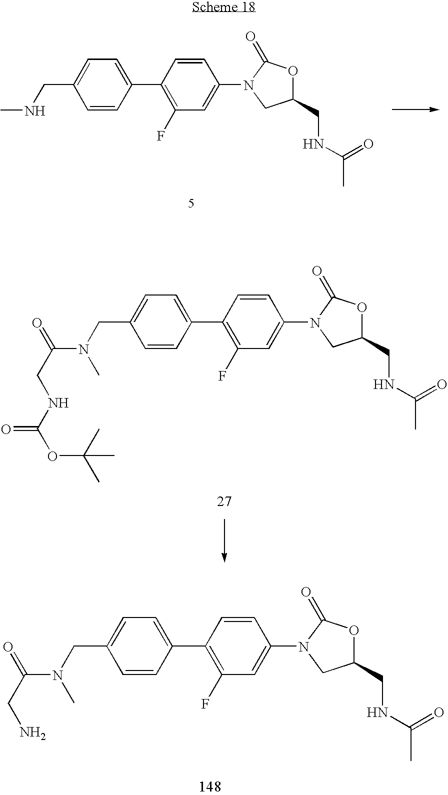

A solution of amine 26 (0.050 g, 0.14 mmol) in DMF (0.50 mL) was treated with N-tert-butoxycarbonyl-glycine (0.026 g, 0.15 mmol), DMAP (0.8 M in DMF, 0.17 mL, 0.14 mmol), and EDCI (0.030 g, 0.15 mmol) and stirred at 23° C. for 0.5 h. The reaction mixture was added directly to a silica column and purified by flash chromatography (50-100% acetone in hexane) to afford 0.031 g amide 27 in 42% yield.

A solution of amide 27 (0.093 g, 0.18 mmol) was treated with hydrogen chloride (4.0 M in dioxane, 1.0 mL) and stirred at 23° C. for 0.5 h. The reaction mixture was diluted with methylene chloride and the solvent removed in vacuo to yield 0.082 g compound 148 in 100% yield. MS (ESI): 429 (M+H)⁺.

### Example 23—Synthesis of Compound 150

A solution of amine 5 (0.18 g, 0.50 mmol) in DMF (2 mL) was treated with acetoxyacetic acid (0.065 g, 0.55 mmol), EDCI (0.11 g, 0.55 mmol) and DMAP (0.061 g, 0.50 mmol), and stirred at 23° C. for 2 h. The reaction mixture was directly purified by flash chromatography (50-100% acetone/hexanes) to provide the O-acetylated product (120 mg, 0.26 mmol, 52%). A solution of the O-acetylated product (0.11 g, 0.24 mmol) in 3:1:1 THF/methanol/H₂O (2.5 mL) was treated with lithium hydroxide monohydrate (0.020 g, 0.48 mmol) and stirred at 23° C. for 15 min. The reaction mixture was diluted with dichloromethane (50 mL), washed with 1 M hydrochloric acid (50 mL), and dried over Na₂SO₄. The solvent was removed in vacuo to provide 90 mg compound 150 in 90% yield as a white powder. MS (ESI): 416 (M+H)⁺.

### Example 24—Synthesis of Compound 157

A solution of mesylate 3 (220 mg, 0.5 mmol, 1 equiv.) in 2 mL of DMF and 2 mL of THF was treated with Hunig's base (0.3 mL, 1.0 mmol, 2 equiv.) and thiosemicarbazide (180 mg, 2.0 mmol, 4.0 equiv.) at room temperature, and the resulting mixture was heated to reflux and stirred for 6 hours. The desired product precipitated from the reaction mixture and was collected by filtration. Purification by column chromatography (5-10% MeOH/CH₂Cl₂ gradient elution) to afford the desired (5S)—N-[3-(4′-N-thiocarbamoylhydrazinomethyl-2-fluoro-biphenyl-4-yl)-2-oxo-oxazolidin-5-ylmethyl]-acetamide 157 (52 mg, 215.5 mg theoretical, 24%) as off-white solids. C₂₀H₂₂FN₅O₃S, LCMS (EI): m/e 432 (M⁺+H).

### Example 25—Synthesis of Compound 170

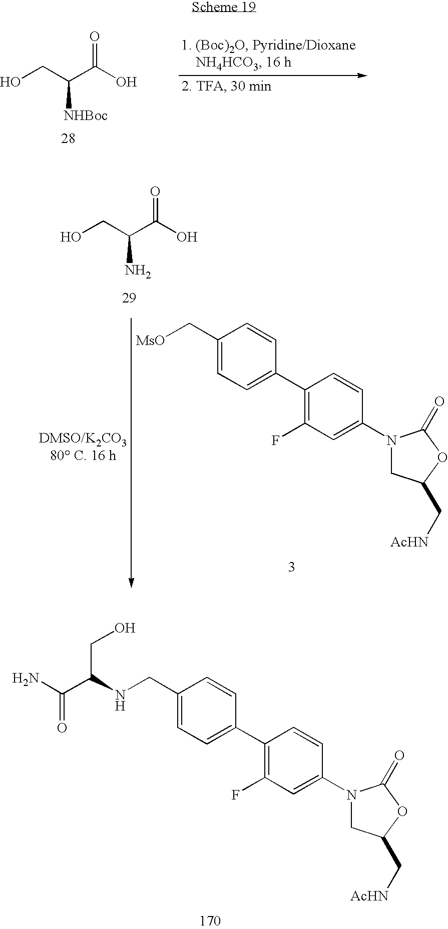

To a solution of Boc-ser-OH 28 (2.0 g, 8.022 mmol) in dioxane (20 mL) and pyridine (4 mL) was added di-tert-butyl dicarbonate (2.1 g, 9.626 mmol). The mixture was stirred at room temperature for 10 min., then ammonium hydrogencarbonate (3.17 g, 40.11 mmol) was added and the reaction mixture was stirred at room temperature for 16 hr. The resulting mixture was diluted with ethyl acetate (50 mL) and washed with saturated aqueous sodium carbonate (50 mL) and saturated sodium chloride (50 mL), dried over Na₂SO₄, and evaporated in vacuo. The residue was dissolved in 5 mL TFA and stirred at room temperature for 30 min. The mixture was concentrated in vacuo and partitioned between 100 mL of ethyl acetate and 100 mL of aqueous sodium carbonate. The mixture was shaken vigorously and the layers allowed to separate. The organic layer was washed with brine (100 mL), dried over anhydrous Na₂SO₄, and concentrated in vacuo to give 0.85 g of amide 29 as a colorless oil. This material was carried on to the next reaction without further purification.

A mixture of mesylate 3 (0.1 g, 0.229 mmol) and amide 29 (0.02 g, 0.229 mmol) in dimethylsulfoxide (5 mL) and K₂CO₃ (0.06 g, 0.458 mmol) was heated at 80° C. for 16 h. The reaction was cooled, diluted with ethyl acetate, and washed with water (50 mL) and brine (50 mL). Drying over Na₂SO₄ and concentration in vacuo afforded a yellow solid, which was purified by preparative thin layer chromatography (95% CH₂Cl₂, 5% MeOH) provided compound 170 as a white solid. LCMS (ESI): 445 (M+H)⁺. ¹HNMR (300 MHz, DMSO-d₆): 8.25 (t, J=5 Hz,1H), 7.61-7.39 (m,7H), 5.25 (s,1H), 4.75 (m, 1H), 4.54 (s, 2H), 4.15 (t, J=9 Hz, 1H), 3.77 (m, 1H), 3.43(d, 2H), 1.8 (s, 3H).

### Example 26—Synthesis of Compound 173

Compound 101 (0.25 g, 0.61 mmol), acetoxyacetic acid 5 (0.09 g, 0.73 mmol) and 1-hydroxybenzotriazole (0.10 g, 0.73 mmol) were dissolved in DMF (8 mL), and to this solution was added EDCI (0.14 g, 0.73 mmol). The mixture was stirred at room temperature for 2 h. The solvent was removed in vacuo and the residue was partitioned between 20% MeOH in CH₂Cl₂ (40 mL) and water (40 mL). The two layers were separated, the aqueous layer was extracted with 20% MeOH in CH₂Cl₂ (3×30 mL), and the combined organic layer was dried over Na₂SO₄. The solvent was removed in vacuo and the crude was purified by silica gel chromatography (1-10% MeOH CH₂Cl₂) to yield compound 173 as a yellow-white solid (0.116 g, 37%). LCMS (ESI): m/e 537.1 (M+Na)⁺.

The entire disclosure of each of the patent documents and scientific articles referred to herein is incorporated by reference for all purposes.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A compound having the formula: 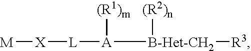
or a pharmaceutically acceptable salt, ester or prodrug thereof, wherein:
A is selected from the group consisting of:
phenyl, pyridyl, pyrazinyl, pyrimidinyl, and pyridazinyl;
B is selected from the group consisting of:
phenyl, pyridyl, pyrazinyl, pyrimidinyl, and pyridazinyl;
Het-CH₂—R³ is selected from the group consisting of: 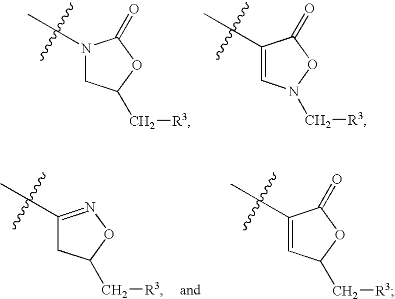
M has the formula: 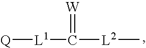
wherein
L¹ is a bond or C₁₋₆ alkyl optionally substituted with one or more R⁴ groups;
L² is a bond or C₁₋₆ alkyl optionally substituted with one or more R⁴ groups;
Q is selected from the group consisting of:
a) H, b) —NR⁴R⁴, c) —OR⁴, and d) C₁₋₆ alkyl optionally substituted with one or more R⁴ groups; and
W is selected from the group consisting of O and S;
X is selected from the group consisting of:
a) —NR⁴—, b) —NR⁴NR⁴—, and c) —S—;
L is C₁₋₆ alkyl optionally substituted with one or more R⁴ groups;
R¹, at each occurrence, independently is selected from the group consisting of:
a) F, b) Cl, c) Br, d) 1, e) —CF₃, f) —OR⁷, g) —CN, h) —NO₂, i) —NR⁷R⁷, j) —C(O)R⁷, k) —C(O)OR⁷, l) —OC(O)R⁷, m) —C(O)NR⁷R⁷, n) —NR⁷C(O)R⁷, o) —OC(O)NR⁷R⁷, p) —NR⁷C(O)R⁷, q) —NR⁷C(O)NR⁷R⁷, r) —C(S)R⁷, s) —C(S)OR⁷, t) —OC(S)R⁷, u) —C(S)NR⁷R⁷, v) —NR⁷C(S)R⁷, w) —OC(S)NR⁷R⁷, x) —NR⁷C(S)OR⁷, y) —NR⁷C(S)NR⁷R⁷, z) —C(NR⁷)R⁷, aa) —C(NR⁷)OR⁷, bb) —OC(NR⁷)R⁷, cc) —C(NR⁷)NR⁷R⁷, dd) —NR⁷C(NR⁷)R⁷, ee) —OC(NR⁷)NR⁷R⁷, ff) —NR⁷C(NR⁷)OR⁷, gg) —NR⁷C(NR⁷)NR⁷R⁷, hh) —S(O)ₚR⁷, ii) —SO₂NR⁷R⁷, and jj) R⁷;
R², at each occurrence, independently is selected from the group consisting of:
a) F, b) Cl, c) Br, d) I, e) —CF₃, f) —OR⁷, g) —CN, h) —NO₂, i) —NR⁷R⁷, j) —C(O)R⁷, k) —C(O)OR⁷, 1)—OC(O)R⁷, m) —C(O)NR⁷R⁷, n) —NR⁷C(O)R⁷, o) —OC(O)NR⁷R⁷, p) —NR⁷C(O)OR⁷, q) —NR⁷C(O)NR⁷R⁷, r) —C(S)R⁷, s) —C(S)OR⁷, t) —OC(S)R⁷, u) —C(S)NR⁷R⁷, v) —NR⁷C(S)R⁷, w) —OC(S)NR⁷R⁷, x) —NR⁷C(S)OR⁷, y) —NR⁷C(S)NR⁷R⁷, z) —C(NR⁷)R⁷, aa) —C(NR⁷)OR⁷, bb) —OC(NR⁷)R⁷, cc) —C(NR⁷)NR⁷R⁷, dd) —NR⁷C(NR⁷)R⁷, ee) —OC(NR⁷)NR⁷R⁷, ff) —NR⁷C(NR⁷)OR⁷, gg) —NR⁷C(NR⁷)NR⁷R⁷, hh) —S(O)ₚR⁷, ii) —SO₂NR⁷R⁷, and jj) R⁷;
R³ is selected from the group consisting of:
a) —OR⁷, b) —NR⁷R⁷, c) —C(O)R⁷, d) —C(O)OR⁷, e) —OC(O)R⁷, f) —C(O)NR⁷R⁷, g) —NR⁷C(O)R⁷, h) —OC(O)NR⁷R⁷, i) —NR⁷C(O)OR⁷, j) —NR⁷C(O)NR⁷R⁷, k) —C(S)R⁷, l) —C(S)OR⁷, m) —OC(S)R⁷, n) —C(S)NR⁷R⁷, o) —NR⁷C(S)R⁷, p) —OC(S)NR⁷R⁷, q) —NR⁷C(S)OR⁷, r) —NR⁷C(S)NR⁷R⁷, s) —C(NR⁷)R⁷, t) —C(NR⁷)OR⁷, u) —OC(NR⁷)R⁷, v) —C(NR⁷)NR⁷R⁷, w) —NR⁷C(NR⁷)R⁷, x) —OC(NR⁷)NR⁷R⁷, y) —NR⁷C(NR⁷)OR⁷, z) —NR⁷C(NR⁷)NR⁷R⁷, aa) —S(O)ₚR⁷, bb) —SO₂NR⁷R⁷, and cc) R⁷;
R⁴, at each occurrence, independently is selected from the group consisting of:
a) H, b) ═O, c) ═S, d) ═NR⁵, e) ═NOR⁵, f) ═N—NR⁵R⁵, g) —OR⁵, h) —NO₂, i) —NR⁵R⁵, j) —C(O)R⁵, k) —C(O)OR⁵, 1)—OC(O)R⁵, m) —C(O)NR⁵R⁵, n) —NR⁵C(O)R⁵, o) —OC(O)NR⁵R⁵, p) —NR⁵C(O)OR⁵, q) —NR⁵C(O)NR⁵R⁵, r) —C(S)R⁵, s) —C(S)OR⁵, t) —OC(S)R⁵, u) —C(S)NR⁵R⁵, v) —NR⁵C(S)R⁵, w) —OC(S)NR⁵R⁵, x) —NR⁵C(S)OR⁵, y) —NR⁵C(S)NR⁵R⁵, z) —C(NR⁵)R⁵, aa) —C(NR⁵)OR, bb) —OC(NR⁵)R⁵, cc) —C(NR⁵)NR⁵R⁵, dd) —NR⁵C(NR⁵)R⁵, ee) —OC(NR⁵)NR⁵R⁵, ff) —NR⁵C(NR⁵)OR⁵, gg) —NR⁵C(NR⁵)NR⁵R⁵, hh) —S(O)ₚR⁵, and ii) R⁵;
R⁵, at each occurrence, independently is selected from the group consisting of:
a) H, b) C₁₋₆ alkyl, c) —C(O)—C₁₋₆ alkyl, and d) —C(O)O—C₁₋₆ alkyl,
wherein any of b)-d) optionally is substituted with one or more R⁶ groups;
R⁶, at each occurrence, independently is selected from the group consisting of:
a) —OH, b) —OC₁₋₆ alkyl, c) —SH, d) —NO₂, e) —NH₂, f) —NHC₁₋₆ alkyl, g) —N(C₁₋₆ alkyl)₂, h) —C(O)H, i) —C(O)OH, j) —C(O)C₁₋₆ alkyl, k) —OC(O)C₁₋₆ alkyl, l) —C(O)OC₁₋₆ alkyl, m) —C(O)NH₂, n) —C(O)NHC₁₋₆ alkyl, o) —C(O)N(C₁₋₆ alkyl)₂, p) —NHC(O)C₁₋₆ alkyl, and q)—S(O)ₚC₁₋₆ alkyl;
R⁷, at each occurrence, independently is selected from the group consisting of:
a) H, b) C₁₋₆ alkyl, c) C₂₋₆ alkenyl, d) C₂₋₆ alkynyl, e) C₃₋₁₄ saturated, unsaturated, or aromatic carbocycle, f) 3-14 membered saturated, unsaturated, or aromatic heterocycle comprising one or more heteroatoms selected from the group consisting of nitrogen, oxygen, and sulfur, g) —C(O)—C₁₋₆ alkyl, h) —C(O)—C₂₋₆ alkenyl, i) —C(O)—C₂₋₆ alkynyl, j) —C(O)—C₃₋₁₄ saturated, unsaturated, or aromatic carbocycle, k) —C(O)-3-14 membered saturated, unsaturated, or aromatic heterocycle comprising one or more heteroatoms selected from the group consisting of nitrogen, oxygen, and sulfur, l) —C(O)O—C₁₋₆ alkyl, m) —C(O)O—C₂₋₆ alkenyl, n) —C(O)O—C₂₋₆ alkynyl, o) —C(O)O—C₃₋₁₄ saturated, unsaturated, or aromatic carbocycle, and p) —C(O)O-3-14 membered saturated, unsaturated, or aromatic heterocycle comprising one or more heteroatoms selected from the group consisting of nitrogen, oxygen, and sulfur, wherein any of b)-p) optionally is substituted with one or more R⁸ groups;
R⁸, at each occurrence, is independently selected from the group consisting of:
a) F, b) Cl, c) Br, d) I, e) ═O, f) ═S, g) ═NR⁹, h) ═NOR⁹, i) ═N—NR⁹R⁹, j) —CF₃, k) —OR⁹, l) —CN, m) —NO₂, n) —NR⁹R⁹, o) —C(O)R⁹, p) —C(O)OR⁹, q) —OC(O)R⁹, r) —C(O)NR⁹R⁹, s) —NR⁹C(O)R⁹, t) —OC(O)NR⁹R⁹, u) —NR⁹C(O)OR⁹, v) —NR⁹C(O)NR⁹R⁹, w) —C(S)R⁹, x) —C(S)OR⁹, y) —OC(S)R⁹, z) —C(S)NR⁹R⁹, aa) —NR⁹C(S)R⁹, bb) —OC(S)NR⁹R⁹, cc) —NR⁹C(S)OR⁹, dd) —NR⁹C(S)NR⁹R⁹, ee) —C(NR⁹)R⁹, ff) —C(NR⁹)OR⁹, gg) —OC(NR⁹)R⁹, hh) —C(NR⁹)NR⁹R⁹, ii) —NR⁹C(NR⁹)R⁹, jj) —OC(NR⁹)NR⁹R⁹, kk) —NR⁹C(NR⁹)OR⁹, ll) —NR⁹C(NR⁹)NR⁹R⁹, m) —S(O)ₚR⁹, m) —SO₂NR⁹R⁹, and oo) R⁹;
R⁹, at each occurrence, independently is selected from the group consisting of:
a) H, b) C₁₋₆ alkyl, c) C₂₋₆ alkenyl, d) C₂₋₆ alkynyl, e) C₃₋₁₄ saturated, unsaturated, or aromatic carbocycle, f) 3-14 membered saturated, unsaturated, or aromatic heterocycle comprising one or more heteroatoms selected from the group consisting of nitrogen, oxygen, and sulfur, g) —C(O)—C₁₋₆ alkyl, h) —C(O)—C₂₋₆ alkenyl, i) —C(O)—C₂₋₆ alkynyl, j) —C(O)—C₃₋₁₄ saturated, unsaturated, or aromatic carbocycle, k) —C(O)-3-14 membered saturated, unsaturated, or aromatic heterocycle comprising one or more heteroatoms selected from the group consisting of nitrogen, oxygen, and sulfur, l) —C(O)O—C₁₋₆ alkyl, m) —C(O)O—C₂₋₆ alkenyl, n) —C(O)O—C₂₋₆ alkynyl, o) —C(O)O—C₃₋₁₄ saturated, unsaturated, or aromatic carbocycle, and p) —C(O)O-3-14 membered saturated, unsaturated, or aromatic heterocycle comprising one or more heteroatoms selected from the group consisting of nitrogen, oxygen, and sulfur, wherein any of b)-p) optionally is substituted with one or more moieties selected from the group consisting of:
a) F, b) Cl, c) Br, d) I, e) —CF₃, f) —OH, g) —OC₁₋₆ alkyl, h) —SH, i) —SC₁₋₆ alkyl, j) —CN, k) —NO₂, l)—NH₂, m) —NHC₁₋₆ alkyl, n) —N(C₁₋₆ alkyl)₂, o) —C(O)C₁₋₆ alkyl, p) —OC(O)C₁₋₆ alkyl, q) —C(O)OC₁₋₆ alkyl, r) —C(O)NH₂, s) —C(O)NHC₁₋₆ alkyl, t) —C(O)N(C₁₋₆ alkyl)₂, u) —NHC(O)C₁₋₆ alkyl, v) —SO₂NH₂—, w) —SO₂NHC₁₋₆ alkyl, x) —SO₂N(C₁₋₆ alkyl)₂, and y) —S(O)ₚC₁₋₆ alkyl;
m is 0, 1, 2, 3, or 4;
n is 0, 1, 2, 3, or 4; and
p, at each occurrence, independently is 0, 1, or 2,
and wherein the compound does not have the formula selected from the group consisting of:

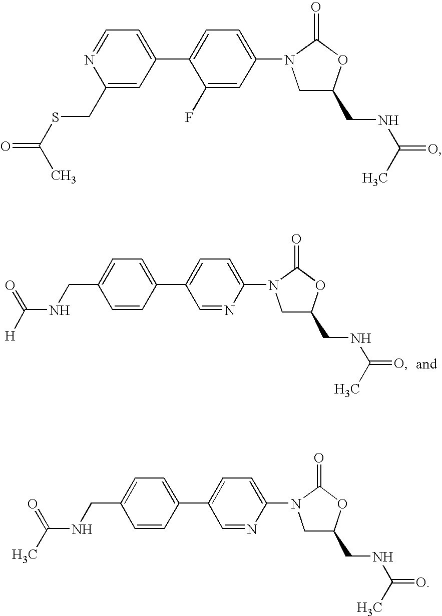

2. The compound according to claim 1, having the formula: 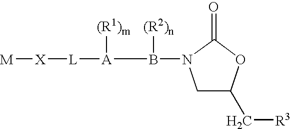
or a pharmaceutically acceptable salt, ester or prodrug thereof,
wherein A, B, L, M, R¹, R², R³, X, m, and n are defined as described in claim 1.

3. The compound according to claim 1, having the formula: 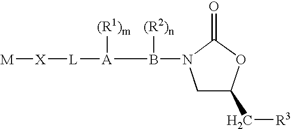
or a pharmaceutically acceptable salt, ester or prodrug thereof,
wherein A, B, L, M, R¹, R², R³, X, m, and n are defined as described in claim 1.

4. The compound according to claim 1, or a pharmaceutically acceptable salt ester or prodrug thereof, wherein
A is selected from the group consisting of phenyl and pyridyl;
B is selected from the group consisting of phenyl and pyridyl;
m is 0, 1, or 2; and
n is 0, 1, or 2.

5. The compound according to 4, or a pharmaceutically acceptable salt, ester or prodrug thereof, wherein A-B is: 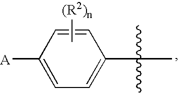
wherein A, R², and n are defined as described in claim 1.

6. The compound according to claim 5, or a pharmaceutically acceptable salt, ester or prodrug thereof, wherein A-B is: 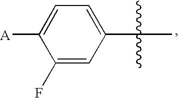
wherein A is defined as described in claim 1.

7. The compound according to claim 5, or a pharmaceutically acceptable salt, ester or prodrug thereof, wherein A-B is: 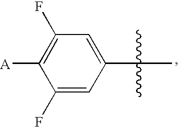
wherein A is defined as described in claim 1.

8. The compound according to claim 1, or a pharmaceutically acceptable salt, ester or prodrug thereof, wherein A-B is: 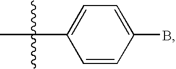
wherein B is defined as described in claim 1.

9. The compound according to claim 1, or a pharmaceutically acceptable salt, ester or prodrug thereof, wherein A-B is: 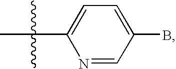
wherein B is defined as described in claim 1.

10. The compound according to claim 1, or a pharmaceutically acceptable salt, ester or prodrug thereof, wherein R³ is —NHC(O)R⁷.

11. The compound according to claim 10, or a pharmaceutically acceptable salt, ester or prodrug thereof, wherein R³ is —NHC(O)CH₃.

12. The compound according to claim 1, or a pharmaceutically acceptable salt, ester or prodrug thereof, wherein R³ is:

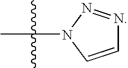

13. The compound according to claim 1 or 2, having the formula: 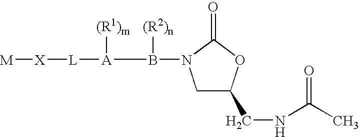
or a pharmaceutically acceptable salt, ester or prodrug thereof,
wherein A, B, L, M, R¹, R², X, m, and n are defined as described in claim 1.

14. The compound according to claim 1, having the formula: 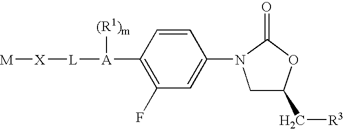
or a pharmaceutically acceptable salt, ester or prodrug thereof,
wherein A, L, M, R¹, R³, X, and m are defined as described in claim 1.

15. The compound according to claim 14, having the formula: 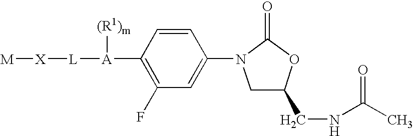
or a pharmaceutically acceptable salt, ester or prodrug thereof,
wherein A, L, M, R¹, X, and m are defined as described in claim 1.

16. The compound according to claim 14, having the formula: 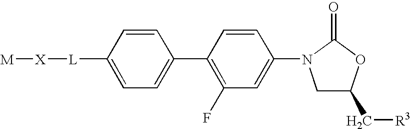
or a pharmaceutically acceptable salt, ester or prodrug thereof,
wherein L, M, R³, and X are defined as described in claim 1.

17. The compound according to claim 16, having the formula: 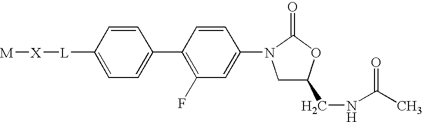
or a pharmaceutically acceptable salt, ester or prodrug thereof,
wherein L, M, and X are defined as described in claim 1.

18. The compound according to claim 14, having the formula: 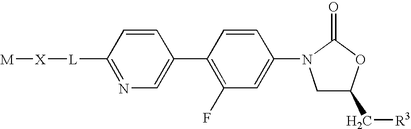
or a pharmaceutically acceptable salt, ester or prodrug thereof,
wherein L, M, R³, and X are defined as described in claim 1.

19. The compound according to claim 18, having the formula: 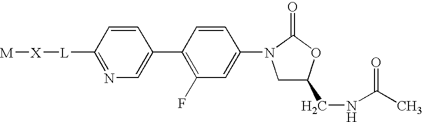
or a pharmaceutically acceptable salt, ester or prodrug thereof,
wherein L, M, and X are defined as described in claim 1.

20. The compound according to claim 1, having the formula: 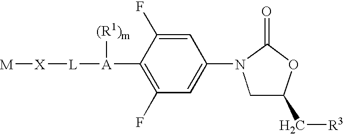
or a pharmaceutically acceptable salt, ester or prodrug thereof,
wherein A, L, M, R¹, R³, X, and m are defined as described in claim 1.

21. The compound according to claim 20, having the formula: 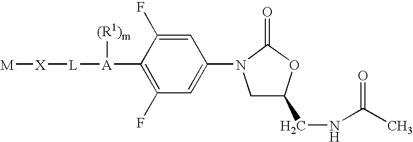
or a pharmaceutically acceptable salt, ester or prodrug thereof,
wherein A, L, M, R¹, X, and m are defined as described in claim 1.

22. The compound according to claim 20, having the formula: 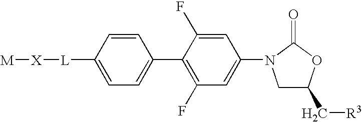
or a pharmaceutically acceptable salt, ester or prodrug thereof,
wherein L, M, R³, and X are defined as described in claim 1.

23. The compound according to claim 22, having the formula: 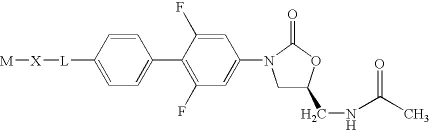
or a pharmaceutically acceptable salt, ester or prodrug thereof,
wherein L, M, and X are defined as described in claim 1.

24. The compound according to claim 20, having the formula: 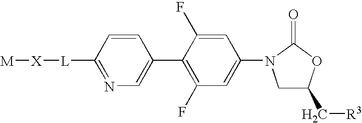
or a pharmaceutically acceptable salt, ester or prodrug thereof,
wherein L, M, R³, and X are defined as described in claim 1.

25. The compound according to claim 24, having the formula: 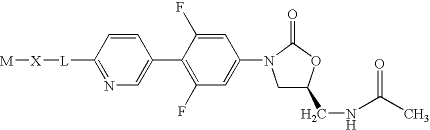
or a pharmaceutically acceptable salt, ester or prodrug thereof,
wherein L, M, and X are defined as described in claim 1.

26. The compound according to claim 1, or a pharmaceutically acceptable salt, ester or prodrug thereof, wherein M is: 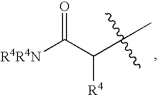
and R⁴, at each occurrence, independently is defined as described in claim 1.

27. The compound according to claim 26, or a pharmaceutically acceptable salt, ester or prodrug thereof, wherein M is: 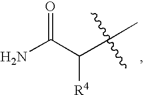
and R⁴ is defined as described in claim 1.

28. The compound according to claim 1, or a pharmaceutically acceptable salt, ester or prodrug thereof, wherein M is: 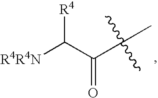
and R⁴, at each occurrence, independently is defined as described in claim 1.

29. The compound according to claim 28, or a pharmaceutically acceptable salt, ester or prodrug thereof, wherein M is: 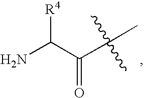
and R⁴ is defined as described in claim 1.

30. The compound according to claim 1, or a pharmaceutically acceptable salt, ester or prodrug thereof, wherein X is —NH—.

31. The compound according to claim 1, or a pharmaceutically acceptable salt, ester or prodrug thereof, wherein X is:

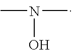

32. A compound having the structure corresponding to any one of the structures listed in Table 1, or a pharmaceutically acceptable salt, ester, or prodrug thereof.

33. A pharmaceutical composition comprising one or more compounds according to claim 1 and a pharmaceutically acceptable carrier.

34. A method of treating a microbial infection in a mammal comprising the step of administering to the mammal an effective amount of one or more compounds according to claim 1.

35. A method of treating a fungal infection in a mammal comprising the step of administering to the mammal an effective amount of one or more compounds according to claim 1.

36. A method of treating a parasitic disease in a mammal comprising the step of administering to the mammal an effective amount of one or more compounds according to claim 1.

37. A method of treating a proliferative disease in a mammal comprising the step of administering to the mammal an effective amount of one or more compounds according to claim 1.

38. A method of treating a viral infection in a mammal comprising the step of administering to the mammal an effective amount of one or more compounds according to claim 1.

39. A method of treating an inflammatory disease in a mammal comprising the step of administering to the mammal an effective amount of one or more compounds according to claim 1.

40. A method of treating a gastrointestinal motility disorder in a mammal comprising the step of administering to the mammal an effective amount of one or more compounds according to claim 14.

41. A method of treating a disorder in a mammal comprising the step of administering to the mammal an effective amount of one or more compounds according to claim 1 thereby to ameliorate a symptom of the disorder, wherein the disorder is selected from the group consisting of:
a skin infection, nosocomial pneumonia, post-viral pneumonia, an abdominal infection, a urinary tract infection, bacteremia, septicemia, endocarditis, an atrio-ventricular shunt infection, a vascular access infection, meningitis, surgical prophylaxis, a peritoneal infection, a bone infection, a joint infection, a methicillin-resistant Staphylococcus aureus infection, a vancomycin-resistant Enterococci infection, a linezolid-resistant organism infection, and tuberculosis.

42. The method according to claim 34, wherein the compound is administered orally, parentally, or topically.

43. A method of synthesizing a compound according to claim 1.

44. A medical device containing one or more compounds according to claim 1.

45. The medical device according to claim 44, wherein the device is a stent.
